(19) United States
(12) Patent Application Publication
(10) Pub. No.: US 2018/0127831 A1
(43) Pub. Date: May 10, 2018

(54) PROGNOSTIC MARKERS OF ACUTE MYELOID LEUKEMIA SURVIVAL

(71) Applicants: UNIVERSITÉ DE MONTRÉAL, MONTREAL (CA); RSEM, LIMITED PARTNERSHIP, MONTREAL (CA)

(72) Inventors: JOSÉE HÉBERT, VILLE MONT-ROYAL (CA); GUY SAUVAGEAU, MONTREAL (CA); VINCENT-PHILIPPE LAVALLÉE, MONTREAL (CA)

(21) Appl. No.: 15/575,890

(22) PCT Filed: May 19, 2016
(86) PCT No.: PCT/CA2016/050562

§ 371 Date: Nov. 21, 2017

Related U.S. Application Data
(60) Provisional application No. 62/164,897, filed on May 21, 2015.

(51) Int. Cl. C12Q 1/6886

(52) U.S. Cl. CPC C12Q 1/6886; C12Q 2600/158

(57) ABSTRACT

Methods and kits for the diagnosis and prognosis of acute myeloid leukemia (AML) are described. These methods and kits are based on the assessment of the level of expression of the gene High Mobility Group AT-hook 2 (HMGA2), and optionally of the level of expression of at least one additional prognostic marker gene such as PRKC Apoptosis WT1 Regulator (PAWR), in a biological sample from an AML patient. High levels of expression of HMGA2 and PAWR in the sample are associated with poor disease prognosis, for example low probability of survival and/or increased risk of relapse, in AML patients, including in intermediate-risk AML patients. Methods and kits for the diagnosis of AMLs with TP53 mutations based on genes differentially expressed in AMLs with TP53 mutations relative to other AMLs also described.

### CROSS REFERENCE TO RELATED APPLICATIONS

The present application claims the benefits of U.S. provisional application Ser. No. 62/164,897, filed on May 21, 2015, which is incorporated herein by reference in its entirety.

### TECHNICAL FIELD

The present invention generally relates to acute myeloid leukemia (AML), and more particularly to the prognosis of AML.

### BACKGROUND ART

Acute Myeloid Leukemia (AML) is a particularly lethal form of cancer, with most patients dying within two years of diagnosis. It is one of the leading causes of death among young adults. AML is a collection of neoplasms with heterogeneous pathophysiology, genetics and prognosis. Mainly based on cytogenetics and molecular analysis, AML patients are presently classified into groups or subsets of AML with markedly contrasting prognosis. Approximately 45% of all AML patients are currently classified into distinct groups with variable prognosis based on the presence or absence of specific recurrent cytogenetic abnormalities. A significant proportion of patients, however, are classified as intermediate-risk AML patients, accounting for approximately 55% of all AML patients. Existing AML prognostic tests are often inaccurate, leaving hemato-oncologists with a lack of tools to guide their decision making about treatment options (e.g., consolidation chemotherapy or allogeneic hematopoietic stem cell transplantation).

Thus, there is a need for the identification of markers for the prognosis of AMLs, i.e. to better predict treatment outcome and/or survival.

The present description refers to a number of documents, the content of which is herein incorporated by reference in their entirety.

### SUMMARY OF THE INVENTION

In a first aspect, the present invention provides the following items 1 to 54:

1. A method for the disease prognosis of a patient suffering from acute myeloid leukemia (AML), said method comprising: measuring the level of expression of High Mobility Group AT-hook 2 (HMGA2) in a biological sample comprising leukemic cells from said patient; and comparing said level of expression to a threshold reference level, wherein a level of expression that is higher than said threshold reference level is indicative of a poor disease prognosis.
2. The method of item 1, wherein said level of expression is measured at the nucleic acid level.
3. The method of item 2, wherein said method comprises amplifying a nucleic acid encoding HMGA2 using a first HMGA2 primer and a second HMGA2 primer.
4. The method of item 3, wherein said first HMGA2 primer comprises at least 10 nucleotides, for example at least 10 contiguous nucleotides, of the sequence 5′-CACTTCAGCCCAGGGACAA-3′ (SEQ ID NO: 1).
5. The method of item 4, wherein said first HMGA2 primer comprises the sequence 5′-CACTTCAGCCCAGGGACAA-3′ (SEQ ID NO: 1).
6. The method of any one of items 3 to 5, wherein said second HMGA2 primer comprises at least 10 nucleotides, for example at least 10 contiguous nucleotides, of the sequence 5′-CTCACCGGTTGGTTCTTGCT-3′ (SEQ ID NO: 2).
7. The method of item 6, wherein said second HMGA2 primer comprises the sequence 5′-CTCACCGGTTGGTTCTTGCT-3′ (SEQ ID NO: 2).
8. The method of any one of items 2 to 7, wherein said method comprises detecting the nucleic acid encoding HMGA2 using a HMGA2 probe.
9. The method of item 8, wherein said HMGA2 probe comprises at least about 10 nucleotides, for example at least 10 contiguous nucleotides, of the sequence 5′-CTCAGAAGAGAGGACGCGGCC-3′ (SEQ ID NO: 3).
10. The method of item 9, wherein said HMGA2 probe comprises the sequence 5′-CTCAGAAGAGAGGACGCGGCC-3′ (SEQ ID NO: 3).
11. The method of any one of items 2 to 10, wherein the level of expression of HMGA2 is measured by reverse transcription polymerase chain reaction (RT-PCR) or RT-qPCR.
12. The method of any one of items 1 to 11, wherein said method further comprises normalizing the level of expression of HMGA2 based on the level of expression of a housekeeping gene.
13. The method of item 12, wherein said housekeeping gene is ABL1.
14. The method of item 13, wherein said method comprises amplifying a nucleic acid encoding ABL1 using a first ABL1 primer and a second ABL1 primer.
15. The method of item 14, wherein said first ABL1 primer comprises at least 10 nucleotides, for example at least 10 contiguous nucleotides, of the sequence 5′-TGGAGATAACACTCTAAGCATAACTAAAGGT-3′ (SEQ ID NO: 4).
16. The method of item 15, wherein said first ABL1 primer comprises the sequence 5′-TGGAGATAACACTCTAAGCATAACTAAAGGT-3′ (SEQ ID NO: 4).
17. The method of any one of items 14 to 16, wherein said second ABL1 primer comprises at least 10 nucleotides, for example at least 10 contiguous nucleotides, of the sequence 5′-GATGTAGTTGCTTGGGACCCA-3′ (SEQ ID NO: 5).
18. The method of item 17, wherein said second ABL1 primer comprises the sequence 5′-GATGTAGTTGCTTGGGACCCA-3′ (SEQ ID NO: 5).
19. The method of any one of items 12 to 18, wherein said method comprises detecting the nucleic acid encoding ABL1 using an ABL1 probe.
20. The method of item 19, wherein said ABL1 probe comprises at least about 10 nucleotides, for example at least 10 contiguous nucleotides, of the sequence 5′-CCATTTTTGGTTTGGGCTTCACACCATT-3′ (SEQ ID NO: 6).
21. The method of item 20, wherein said ABL1 probe comprises the sequence 5′-CCATTTTTGGTTTGGGCTTCACACCATT-3′ (SEQ ID NO: 6).
22. The method of any one of items 1 to 21, further comprising measuring the level of expression of at least one additional prognostic marker gene in said biological sample.
23. The method of item 22, wherein said at least one additional prognostic marker gene is PRKC Apoptosis WT1 Regulator (PAWR).
24. The method of item 23, wherein said method comprises amplifying a nucleic acid encoding PAWR using a first PAWR primer and a second PAWR primer.
25. The method of item 24, wherein said first PAWR primer comprises at least 10 nucleotides, for example at least 10 contiguous nucleotides, of the sequence 5′-TGGTCAACATCCCTGCCG-3′ (SEQ ID NO: 7).
26. The method of item 25, wherein said first PAWR primer comprises the sequence 5′-TGGTCAACATCCCTGCCG-3′ (SEQ ID NO: 7).
27. The method of any one of items 24 to 26, wherein said second PAWR primer comprises at least 10 nucleotides, for example at least 10 contiguous nucleotides, of the sequence 5′-TTGCATCTTCTCGTTTCCGC-3′ (SEQ ID NO: 8).
28. The method of item 27, wherein said second PAWR primer comprises the sequence 5′-TTGCATCTTCTCGTTTCCGC-3′ (SEQ ID NO: 8).
29. The method of any one of items 22 to 28, wherein said method comprises detecting the nucleic acid encoding PAWR using a PAWR probe.
30. The method of item 29, wherein said PAWR probe comprises at least 10 nucleotides, for example at least 10 contiguous nucleotides, of the sequence 5′-AGTACGAAGATGATGAAGCAGGGC-3′ (SEQ ID NO: 9).
31. The method of item 30, wherein said PAWR probe comprises the sequence 5′-AGTACGAAGATGATGAAGCAGGGC-3′ (SEQ ID NO: 9).
32. The method of any one of items 23 to 31, wherein said method further comprises normalizing the level of expression of PAWR based on the level of expression of a housekeeping gene.
33. The method of item 32, wherein said housekeeping gene is ABL1.
34. The method of item 33, wherein said normalization is performed according to the method defined in any one of items 14 to 21.
35. The method of any one of items 1 to 34, wherein said biological sample comprises nucleic acids obtained from peripheral blood cells or bone marrow cells.
36. The method of any one of items 1 to 35, wherein said poor disease prognosis comprises low probability of survival.
37. The method of any one of items 1 to 36, wherein said AML is an intermediate-risk AML.
38. The method of item 37, wherein said intermediate-risk is FLT3-ITD negative AML.
39. The method of any one of items 1 to 38, wherein said patient is less than 60 years old.
40. A method for determining the likelihood that a subject suffers from TP53-mutant acute myeloid leukemia (TP53mut AML), said method comprising:
(i) determining the level of expression of at least one of the genes listed in Table 2A and/or Table 2B in a biological sample comprising leukemic cells from said subject:
(ii) comparing said level of expression to a reference level of expression; and
(iii) determining the likelihood that said subject suffers from TP53mut AML based on said comparison;
   wherein a differential expression of said at least one gene in said biological sample relative to said reference level of expression is indicative that said subject has a high likelihood of suffering from TP53mut AML.
   41. The method of item 39, wherein said method comprises determining the level of expression of at least one of the genes listed in Table 2A in said biological sample, and wherein a higher expression of said at least one gene in said sample relative to said reference level of expression is indicative that said subject has a high likelihood of suffering from TP53mut AML.
   42. The method of item 39 or 40, wherein said the level of expression is measured at the nucleic acid level.
   43. The method of item 41, wherein said the level of expression is measured by RNA sequencing (RNAseq) or reverse transcription polymerase chain reaction (RT-PCR), for example quantitative RT-PCR (RT-qPCR).
   44. A method for treating an AML patient having a good or poor disease prognosis identified based on expression of HMGA2 in a biological sample comprising leukemic cells from said patient, said method comprising treating said patient with a suitable treatment regimen for good or poor prognosis AML.
   45. The method of item 44, wherein said AML patient having a good or poor disease prognosis is further identified based on expression of PAWR in said biological sample.
   46. The method of item 44, wherein said method further comprises performing the method defined in any one of items 1 to 39 to identify said AML patient having a good or poor disease prognosis.
   47. The method of any one of items 44 to 46, wherein said patient has poor disease prognosis, and wherein said treatment regimen comprises stem cell or bone marrow transplantation.
   48. An assay mixture for the prognosis of AML, the assay mixture comprising: (i) a biological sample comprising leukemic cells from a patient suffering from AML; and (ii) one or more reagents for measuring the level of expression of HMGA2 in the sample.
   49. The assay mixture of item 48, further comprising (iii) one or more reagents for measuring the level of expression of PAWR in the sample.
   50. The assay mixture of item 48 or 49, wherein said one or more reagents for measuring the level of expression of HMGA2 in the sample comprises one or more of the primers and/or probes defined in any one of items 4 to 10.
   51. The assay mixture of item 49 or 50, wherein said one or more reagents for measuring the level of expression of PAWR in the sample comprises one or more of the primers and/or probes defined in any one of items 25 to 31.
   52. The assay mixture of any one of items 48 to 51, further comprising (iv) one or more reagents for measuring the level of expression of a housekeeping gene in the sample.
   53. The assay mixture of item 52, wherein said housekeeping gene is ABL1.
   54. The assay mixture of item 52, wherein said one or more reagents for measuring the level of expression of ABL1 in the sample comprises one or more of the primers and/or probes defined in any one of items 15 to 21.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

In the appended drawings:

FIGS. 1A to 1C show the approach used to identify the markers described herein. FIG. 1A: A total of 433 primary human AML specimens were subjected to a refined process to identify novel indications of prognostic/diagnostic value. Briefly, mutations, classical cytogenetic groups, novel fusions and the transcriptomic signatures are first identified to separate AML specimens into appropriate subtypes. A comparison between the transcriptomes of TP53 mut AML (associated with poor prognosis) and the rest of the cohort identified HMGA2 as amongst the most differentially expressed gene in this subtype. PAWR was previously identified as one of the most differentially expressed gene in the EVI1-r AML genetic subtype (LavaHee V P, et al. Blood. 2015 Jan. 1; 125(1):140-3). FIG. 1B: The expression levels of these genes were subsequently explored in the complete cohort. The resulting scatterplot comparing HMGA2 and PAWR expression in the entire cohort is seen in FIG. 1B, with each dot representing the RPKM of each gene as indicated on the axes. Highlighted points demarcate HMGA2+ and/or PAWR+ samples. This analysis resultantly identified additional samples expressing one or both markers but not associated to TP53 mut or EVI1-r genetic aberrations. FIG. 1C: These new groups (HMGA2+ and/or PAWR+ vs. HMGA2-PAWR−) were further examined via Kaplan-Meier survival curve analysis. As observed in FIG. 1C and further detailed herein, the overall survival curve of this new group was found to associate to adverse outcome. Therefore, this approach is thereby able to identify a significant number of patients with poor prognosis (168 HMGA2+ and/or PAWR+ compared to 55 TP53 mutated or EVI1-rearranged samples).

FIG. 2 shows the correlation between transcriptome data and HMGA2 real-time quantitative RT-PCR (RT-qPCR) assay. Scatter plot shows robust correlation in HMGA2 expression levels detected using either RNA-Seq transcriptome data (log RPKM) or the real-time RT-qPCR assay described herein (normalized copy number, NCN). To avoid issues with log-scale representation of RPKM equal to zero, a small constant (0.0001) was added to all expression values.

FIG. 3A shows AML specimens assessed by the HMGA2 RT-qPCR assay were separated according to normalized values of HMGA2 copy number per 10⁴ ABL1 copy number and subjected to Kaplan-Meier survival analysis. A significant difference in overall survival was observed in samples expressing greater than or equal to 1000 normalized copy numbers. FIG. 3B: Dot plot analysis shows the expression levels of genetic subtypes within the Leucegene AML cohort as determined using the HMGA2 RT-qPCR assay. Each triangle represents HMGA2 expression for one specimen, reported as normalized copy number. The previously identified cut-off at 1000 normalized copy number is indicated by the dotted line. Greater than 1000 normalized copy number expression is associated with genetic subtypes with known adverse clinical outcome, however, several additional specimens are identified with expression above cut-off but are either normal karyotype, intermediate abnormal AML, or otherwise not associated with genetic subgroups of poor clinical outcome. Complex karyotype without TP53 mutation or deletion (TP53 wt), n=20; Complex karyotype with TP53 mutation and/or deletion, n=22; Normal karyotype (NK), n=117; Intermediate abnormal karyotype (Interm.abn.), n=50; Trisomy 8 alone, n=12; MLL fusions, n=27; t(8:21), n=17; inv(16), n=28; EVI1r, n=6; Monosomy 5/5q—or monosomy 7/7q—not complex (NC), n=7; NUP98-NSD1 fusion in AML with normal karyotype (NK), n=5; t(6;9), n=2. Median values are indicated by a horizontal line. Abbreviation: EVI1r, AML with EVI1 rearrangements.

FIGS. 4A and 4B show the overall survival curves according to HMGA2 expression in Leucegene de novo AML Intermediate Risk FLT3-ITD-negative cohort. Kaplan-Meier survival probability analysis on Intermediate Risk FLT3-ITD-negative (ITD−) AML based upon HMGA2 expression determined by RNA-Seq (FIG. 4A) and a RT-qPCR assay (FIG. 4B) shows poor overall survival by specimens whose HMGA2 expression is >0.1 RPKM (FIG. 4A) or 1000 normalized values of HMGA2 copy number per 10⁴ ABL1 copy number (FIG. 4B). Leucegene de novo AML Intermediate Risk cohort includes, intermediate abnormal karyotype, some MLL fusions (e.g., t(9;11)/MLL-MLLT3), Normal Karyotype, NUP98-NSD1 fusion in AML with normal karyotype, and trisomy 8 alone. FLT3-ITD and NUP98-NSD1 status were determined by Next Generation Sequencing (NGS).

FIG. 5 shows the overall survival curves according to HMGA2 expression in the Leucegene AML cohort. Kaplan-Meier survival probability analysis based upon HMGA2 expression determined by the RT-qPCR assay shows poor overall survival by specimens whose HMGA2 expression is 1000 normalized copy number (NCN) vs. specimens<1000 NCN. AML patients with a favourable prognosis (t(8;21) and inv(16)) and for which the HMGA2-PAWR test is less informative (AML with MLL fusions) were excluded from the survival analysis.

FIG. 6 shows the combined PAWR and HMGA2 analysis using RT-qPCR in the Leucegene AML cohort. Combined dot plot analysis showing expression of HMGA2 and PAWR based on RT-qPCR results identifies a cohort with robust expression of at least one of either marker. PAWR (grey triangles) and HMGA2 (black triangles) expression are reported as the normalized values of PAWR or HMGA2 copy number per 10⁴ ABL1 copy number. A cut off was established at 1000 normalized copy number (dotted line). Complex karyotype without TP53 mutation or deletion (TP53 wt), n=20; Complex karyotype with TP53 mutation and/or deletion, n=22; Normal karyotype (NK), n=117; Intermediate abnormal karyotype (Interm.abn.), n=50; Trisomy 8 alone, n=12; MLL fusions, n=27; t(8:21), n=17; inv(16), n=28; EVI1r, n=6; Monosomy 5/5q—or monosomy 7/7q—not complex (NC), n=7; NUP98-NSD1 in AML with normal karyotype (NK), n=5; t(6;9), n=2. Median values are indicated by a horizontal line. Abbreviation: EVI1r, AML with EVI1 rearrangements.

FIGS. 7A to 7C show the overall survival curves according to HMGA2/PAWR expression in Leucegene de novo AML Intermediate Risk FLT3-ITD-negative cohort. Kaplan-Meier survival probability analysis on Intermediate Risk FLT3-ITD-negative (ITD−) AML based upon HMGA2 and/or PAWR expression determined by RNA-Seq (FIG. 7A) and RT-qPCR assay (FIGS. 7B and 7C) shows poor overall survival by specimens whose HMGA2 and/or PAWR expression is >0.1 RPKM or 1 RPKM, respectively (FIG. 7A) or 1000 normalized values of HMGA2/PAWR copy number per 10⁴ ABL1 copy number (FIGS. 7B and 7C). Leucegene de novo AML Intermediate Risk cohort includes, intermediate abnormal karyotype, some MLL fusions (e.g., t(9;11)/MLL-MLLT3), Normal Karyotype, NUP98/NSD1 fusion in AML with normal karyotype, and trisomy 8 alone. FLT3-ITD and NUP98-NSD1 status were determined by Next Generation Sequencing (NGS).

FIGS. 8A and 8B show the overall survival curves based upon combinations of HMGA2 and PAWR expression in Leucegene de novo AML cohort. Kaplan-Meier survival probability analysis based upon HMGA2 and PAWR expression determined by the respective RT-qPCR assays shows the poorest overall survival by specimens double positive for HMGA2 and PAWR expression i.e. 1000 normalized values of HMGA/PAWR copy number per 10⁴ ABL1 copy number (NCN). Specimens deemed to be singly positive in expression 1000 NCN) for either HMGA2 or PAWR are deemed to have worse overall survival compared to specimens<1000 NCN for either marker. In FIG. 8B, AML patients with a favourable prognosis (t(8;21) and inv(16)) and for which the HMGA2-PAWR test is less informative (AML with MLL fusions) were excluded from the survival analysis.

FIGS. 9A to 9C show the cDNA (FIGS. 9A and 9B, SEQ ID NO: 10) and protein (FIG. 9C, SEQ ID NO: 11) sequences of human HMGA2 (transcript variant 1, NCBI Reference Sequence: NM_003483.4). The coding sequence is indicated in bold in FIG. 9A, and the nucleotides corresponding to the primers and probes used in the RT-qPCR experiments described herein are underlined (the hybridized sequence is underlined in the case of the reverse primer).

FIGS. 10A and 10B show the cDNA (FIG. 10A, SEQ ID NO: 12) and protein (FIG. 10B, SEQ ID NO: 13) sequences of human PAWR (NCBI Reference Sequence: NM_002583.2). The coding sequence is indicated in bold in FIG. 10A, and the nucleotides corresponding to the primers and probes used in the RT-PCR experiments described herein are underlined (the hybridized sequence is underlined in the case of the reverse primer).

FIG. 11 shows a comparative scatterplot of expressed genes in TP53-mutant AMLs compared to TP53-wildtype AML. Highlighted are the genes with the greatest differential overexpression (HMGA2) and underexpression (EDA2R) in TP53-mutant AML. To avoid issues with log-scale representation of RPKM equal to zero, a small constant (0.0001) was added to all expression values.

FIGS. 12A-12F show the predictive value of HMGA2 and PAWR prognostic markers on overall survival and cumulative incidence of relapse in AML patients of the test cohort (263 de novo AML samples from the Leucegene cohort and 95 de novo AML samples from an independent BCLQ cohort; all patients in the test cohort were treated with curative intent, 7+3 based regimen). Kaplan-Meier estimates of overall survival (FIGS. 12A, 12C and 12E) and cumulative incidence of relapse (FIGS. 12B, 12D and 12F) according to HMGA2 and PAWR expression values (in NCN). Analyses in the global test cohort (FIGS. 12A-12B), in patients younger than 60 years old (FIGS. 12C-120), and in intermediate genetic risk patients younger than 60 years old (FIGS. 12E-12F) are depicted. HMGA2+, ≥1100 NCN; HMGA2−, <1100 NCN; PAWR+, ≥950 NCN; PAWR−, <950 NCN; NCN, normalized copy number.

### DISCLOSURE OF INVENTION

Terms and symbols of genetics, molecular biology, biochemistry and nucleic acids used herein follow those of standard treatises and texts in the field, e.g. Kornberg and Baker, DNA Replication, Second Edition (W.H. Freeman, New York, 1992); Lehninger, Principles of Biochemistry, Sixth Edition (W.H. Freeman, 2012); Strachan and Read, Human Molecular Genetics, Third Edition (Wiley-Liss, New York, 2004); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like. All terms are to be understood with their typical meanings established in the relevant art.

The articles “a” and “an” are used herein to refer to one or to more than one (i.e. to at least one) of the grammatical object of the article. By way of example, “an element” means one element or more than one element. Throughout this specification, unless the context requires otherwise, the words “comprise,” “comprises” and “comprising” will be understood to imply the inclusion of a stated step or element or group of steps or elements but not the exclusion of any other step or element or group of steps or elements.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

All methods described herein can be performed in any suitable order unless otherwise indicated herein or otherwise clearly contradicted by context.

The use of any and all examples, or exemplary language (“e.g.”, “such as”) provided herein, is intended merely to better illustrate the invention and does not pose a limitation on the scope of the invention unless otherwise claimed.

Herein, the term “about” has its ordinary meaning. The term “about” is used to indicate that a value includes an inherent variation of error for the device or the method being employed to determine the value, or encompass values close to the recited values, for example within 10% or 5% of the recited values (or range of values).

Any and all combinations and subcombinations of the embodiments and features disclosed herein are encompassed by the present invention. For example, any combination of 2, 3, 4, 5 or more of the genes identified herein (e.g., in Table 2A and/or 2B) may be used for the prognosis of AML.

The terms “subject” and “patient” are used interchangeably herein, and refer to an animal, preferably a mammal, most preferably a human. In an embodiment, the patient is less than 60 years old. In another embodiment, the patient is 60 years old or older. In another embodiment, the AML patient is a pediatric AML patient.

In the studies described herein, the present inventors have shown that certain genes show differential expression in AMLs with TP53 mutations (relative to other AML subtypes), and that one of the genes overexpressed in AMLs with TP53 mutations, HMGA2, constitutes a suitable prognostic marker for AMLs (including intermediate-risk AMLs), and that its prognostic value may be improved/increased by combining it with PAWR expression.

Accordingly, in a first aspect, the present invention provides a method for the disease prognosis of a patient suffering from acute myeloid leukemia (AML), said method comprising: measuring the level of expression of one or more of the genes listed in Table 2A in a biological sample from said patient; and comparing said level of expression to a threshold reference level, wherein a level of expression that is higher than said threshold reference level is indicative of a poor disease prognosis.

In another aspect, the present invention provides a method for the disease prognosis of a patient suffering from acute myeloid leukemia (AML), said method comprising: measuring the level of expression of one or more of the genes listed in Table 2B in a biological sample from said patient; and comparing said level of expression to a threshold reference level, wherein a level of expression that is lower than said threshold reference level is indicative of a poor disease prognosis.

In another aspect, the present invention provides a method for the disease prognosis of a patient suffering from acute myeloid leukemia (AML), said method comprising: measuring the level of expression of HMGA2 in a biological sample from said patient; and comparing said level of expression to a threshold reference level, wherein a level of expression that is higher than said threshold reference level is indicative of a poor disease prognosis.

In an embodiment, the method further comprises measuring the level of expression of an additional prognostic marker, for example PAWR, in the biological sample from said patient.

In another aspect, the present invention provides a method for the disease prognosis of a patient suffering from acute myeloid leukemia (AML), said method comprising: measuring the levels of expression of HMGA2 and PAWR in a biological sample from said patient; and comparing said levels of expression to threshold reference levels, wherein a level of expression of (i) HMGA2 or (ii) HMGA2 and PAWR that is/are above the threshold reference level(s) is indicative of a poor disease prognosis. In an embodiment, the levels of expression of both HMGA2 and PAWR are above the threshold reference levels.

In an embodiment, the above-mentioned method is used for the prognosis of intermediate-risk AML, which includes for example normal karyotype (NK) AML, trisomy 8 alone AML, intermediate abnormal karyotype AML and wild-type NPM1 without FLT3-ITD and cytogenetic abnormalities not classified as favorable or adverse (see, e.g., Döhner H et al., Blood 115(3):453-474; Dohner and Paschka, American Society of Hematology Education Book, vol. 2014, no. 1: 34-43). In a further embodiment, the intermediate-risk AML is intermediate Risk FLT3-ITD negative AML. The term “Intermediate risk AML” (also referred to as “intermediate-risk cytogenetic subclass of AML”) is commonly used to refer to AML without favorable and particular unfavorable cytogenetic aberrations (i.e. “uninformative” cytogenetic aberrations), and account for a significant proportion (approximately 55%) of AML patients. In an embodiment, the patient does not suffer from AML with MLL fusions. In an embodiment, the intermediate risk AML comprises intermediate cytogenetics, biallelic CEBPA gene mutations and FLT3-ITD negative AML. In an embodiment, the above-mentioned method further comprises identifying or selecting an AML patient having intermediate-risk AML (e.g., based on risk stratification methods/parameters such as cytogenetic aberrations and mutational status) prior to measuring the levels of expression of HMGA2, and PAWR in a sample from said AML patient.

As used herein, the term “prognosis” refers to the forecast of the probable outcome or course of AML; the patient's chance of survival. Accordingly, a less favorable, negative or poor prognosis is defined by a lower post-treatment survival term or survival rate, e.g., decreased survival rate, higher risk of relapse of the AML, higher likelihood of being refractory to induction therapy and/or lower likelihood of achieving a complete remission. Conversely, a positive, favorable, or good prognosis is defined by an elevated post-treatment survival term or survival rate, e.g., increased survival rate, lower risk of relapse of the AML, lower likelihood of being refractory to induction therapy and/or higher likelihood of achieving a complete remission. Survival is usually calculated as an average number of months (or years) that 50% of patients survive, or the percentage of patients that are alive after 1, 2, 3, 4, 5, 10 years, etc. Prognosis is important for treatment decisions because patients with a good prognosis are usually offered less invasive/aggressive treatments (e.g., standard consolidation chemotherapy), while patients with poor prognosis are usually offered more aggressive treatment, such as stem cell/bone marrow transplantation, and/or any other aggressive treatment. In an embodiment, the poor disease prognosis comprises poor overall survival, for example a low likelihood (e.g., less than about 50, 40, 30, 20%) of survival over a period of 1, 2, 3, 4 or 5 years.

As used herein, the term “diagnosis” refers to the assessment of whether a subject suffers from the disease or not, e.g., for determining the likelihood that the patient suffers from the disease. As will be understood by those skilled in the art, such an assessment is usually not intended to be correct for all (i.e. 100%) of the subjects to be identified. The term, however, means that a statistically significant portion of subjects can be identified (e.g. a cohort in a cohort study), which may be determined by the person skilled in the art using various well known statistic evaluation tools, e.g., determination of confidence intervals, p-value determination, Student's t-test, Mann-Whitney test etc. (see, e.g., Dowdy and Wearden, Statistics for Research, Third Edition, John Wiley & Sons, New York, 2004). Preferred confidence intervals are at least 90%>, at least 95%, at least 97%, at least 98% or at least 99%. The p-values are, preferably, 0.1, 0.05, 0.01, 0.005, or 0.0001. More preferably, at least 60%, at least 70%, at least 80% or at least 90% of the subjects of a population can be properly identified by the method of the present invention. Diagnosis according to the present invention includes applications of the method in monitoring, confirmation, and sub-classification of the AML.

In an embodiment, the method of prognosis described herein is performed at different time points in a patient, e.g. to monitor AML progression, treatment efficacy, etc.

In another aspect, the present invention relates to a method for determining the likelihood that a subject suffers from TP53-mutant acute myeloid leukemia (TP53mut AML), said method comprising: determining/measuring the level of expression of at least one of the genes listed in Table 2A and/or Table 2B in a leukemia cell sample (blood cell or bone marrow sample) from said subject: comparing said level of expression to a control/reference level of expression (e.g., expression in a non-TP53mut AML (or wild-type TP53 AML) leukemia sample and/or a normal CD34+ cell sample) and determining the likelihood that said subject suffers from TP53mut AML based on said comparison, wherein a differential expression of said at least one gene in said sample relative to said control sample is indicative that said subject has a high likelihood of suffering from TP53mut AML.

In another aspect, the present invention relates to a method for determining the likelihood that a subject suffers from TP53mut AML, said method comprising: determining/measuring the level of expression of at least one of the genes listed in Table 2A in a leukemia cell sample from said subject: wherein a higher expression of said at least one gene in said sample relative to a control non-TP53mut AML sample is indicative that said subject has a high likelihood of suffering from TP53mut AML.

In another aspect, the present invention relates to a method for determining the likelihood that a subject suffers from TP53mut AML, said method comprising: determining/measuring the level of expression of at least one of the genes listed in Table 2B in a leukemia cell sample from said subject: wherein a lower expression of said at least one gene in said sample relative to a control non-TP53mut AML sample is indicative that said subject has a high likelihood of suffering from TP53mut AML.

The levels of nucleic acids corresponding to the genes identified herein (e.g., those listed in Tables 2A and 2B), for example HMGA2, and the additional prognostic marker (e.g., PAWR) may be evaluated according to the methods disclosed below, e.g., with or without the use of nucleic acid amplification methods. In some embodiments, nucleic acid amplification methods can be used to detect the level of expression of to the genes identified herein (e.g., those listed in Tables 2A and 2B), for example HMGA2, and the additional prognostic marker (e.g., PAWR). For example, the oligonucleotide primers and probes may be used in amplification and detection methods that use nucleic acid substrates isolated by any of a variety of well-known and established methodologies (e.g., Sambrook et al., Molecular Cloning, A Laboratory Manual, pp. 7.37-7.57 (2nd ed., 1989); Lin et al., in Diagnostic Molecular Microbiology, Principles and Applications, pp. 605-16 (Persing et al., eds. (1993); Ausubel et al., Current Protocols in Molecular Biology (2001 and later updates thereto)). Methods for amplifying nucleic acids include, but are not limited to, for example the polymerase chain reaction (PCR) and reverse transcription PCR (RT-PCR), including quantitative RT-PCR (see e.g., U.S. Pat. Nos. 4,683,195; 4,683,202; 4,800,159; 4,965,188), ligase chain reaction (LCR) (see, e.g., Weiss, Science 254: 1292-93 (1991)), strand displacement amplification (SDA) (see e.g., Walker et al., Proc. Natl. Acad. Sci. USA 89:392-396 (1992); U.S. Pat. Nos. 5,270,184 and 5,455,166), Thermophilic SDA (tSDA) (see e.g., European Pat. No. 0 684 315) and methods described in U.S. Pat. No. 5,130,238; Lizardi et al., BioTechnol. 6:1197-1202 (1988); Kwoh et al., Proc. Natl. Acad. Sci. USA 86:1173-77 (1989); Guatelli et al., Proc. Natl. Acad. Sci. USA 87:1874-78 (1990); U.S. Pat. Nos. 5,480,784; 5,399,491; U.S. Publication No. 2006/46265. The methods include the use of Transcription Mediated Amplification (TMA), which employs an RNA polymerase to produce multiple RNA transcripts of a target region (see, e.g., U.S. Pat. Nos. 5,480,784; 5,399,491 and U.S. Publication No. 2006/46265). The levels of nucleic acids may also be measured by “Next Generation Sequencing” (NGS) methods such as RNA sequencing, as well as digital polymerase chain reaction (digital PCR, dPCR) (see, e.g., Pohl, G; Shih, I-M (2004). Expert Review of Molecular Diagnostics (Informa) 4 (1): 41-7) or Nanostring Technology (see, e.g., U.S. Pat. No. 7,919,237; Kulkarni, Meghana M. (2011). Current Protocols in Molecular Biology. 2513.10.1-256.10.17).

Polymerase chain reaction (PCR) is carried out in accordance with known techniques. See, e.g., U.S. Pat. Nos. 4,683,195; 4,683,202; 4,800,159; and 4,965,188. In general, PCR involves, a treatment of a nucleic acid sample (e.g., in the presence of a heat stable DNA polymerase) under hybridizing conditions, with one oligonucleotide primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analyzed to assess the level of expression of the nucleic acid of interest (HMGA2, PAWR). Detection of the amplified sequence may be carried out by visualization following Ethidium Bromide (EtBr) staining of the DNA following gel electrophoresis, or using a detectable label in accordance with known techniques, and the like. For a review on PCR techniques (see PCR Protocols, A Guide to Methods and Amplifications, Michael et al. Eds, Acad. Press, 1990).

Ligase chain reaction (LCR) is carried out in accordance with known techniques (Weiss, 1991, Science 254:1292). Adaptation of the protocol to meet the desired needs can be carried out by a person of ordinary skill. Strand displacement amplification (SDA) is also carried out in accordance with known techniques or adaptations thereof to meet the particular needs (Walker et al., 1992, Proc. Natl. Acad. Sci. USA 89:392-396; and ibid., 1992, Nucleic Acids Res. 20:1691-1696).

“Nucleic acid hybridization” refers generally to the hybridization of two single-stranded nucleic acid molecules having complementary base sequences, which under appropriate conditions will form a thermodynamically favored double-stranded structure. Examples of hybridization conditions can be found in the two laboratory manuals referred above (Sambrook et al., 1989, supra and Ausubel, et al. (eds), 1989, Current Protocols in Molecular Biology, Vol. 1, Green Publishing Associates, Inc., and John Wiley & Sons, Inc., New York,) and are commonly known in the art. Hybridization to filter-bound sequences under moderately stringent conditions may, for example, be performed in 0.5 M NaHPO₄, 7% sodium dodecyl sulfate (SDS), 1 mM EDTA at 65° C., and washing in 0.2×SSC/0.1% SDS at 42° C. (see Ausubel, et al. (eds), 1989, Current Protocols in Molecular Biology, Vol. 1, Green Publishing Associates, Inc., and John Wiley & Sons, Inc., New York, at p. 2.10.3). Alternatively, hybridization to filter-bound sequences under stringent conditions may, for example, be performed in 0.5 M NaHPO₄, 7% SDS, 1 mM EDTA at 65° C., and washing in 0.1×SSC/0.1% SDS at 68° C. (see Ausubel, et al. (eds), 1989, supra). In other examples of hybridization, a nitrocellulose filter can be incubated overnight at 65° C. with a labeled probe specific to one or the other two alleles in a solution containing 50% formamide, high salt (5×SSC or 5×SSPE), 5×Denhardt's solution, 1% SDS, and 100 μg/ml denatured carrier DNA (i.e. salmon sperm DNA). The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency). Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York). The selected temperature is based on the melting temperature (Tm) of the DNA hybrid (Sambrook et al. 1989, supra). Generally, stringent conditions are selected to be about 5° C. lower than the thermal melting point for the specific sequence at a defined ionic strength and pH.

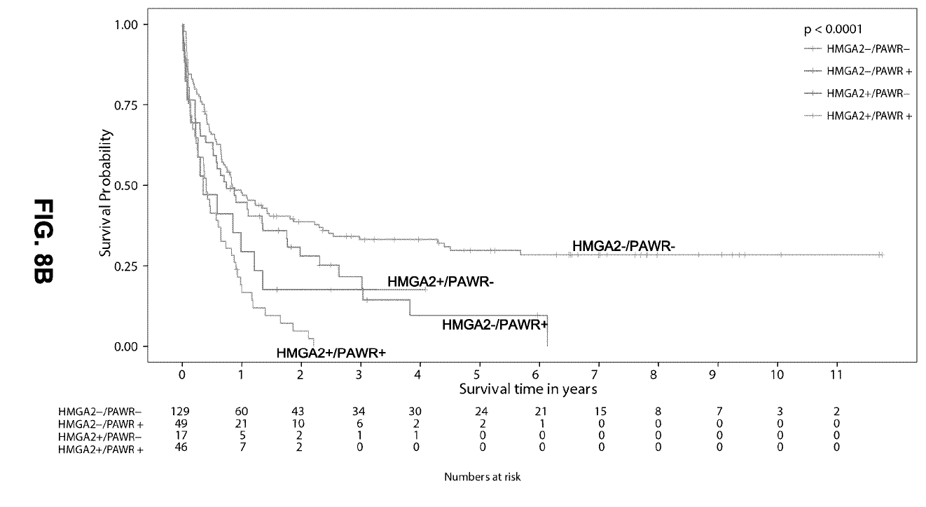

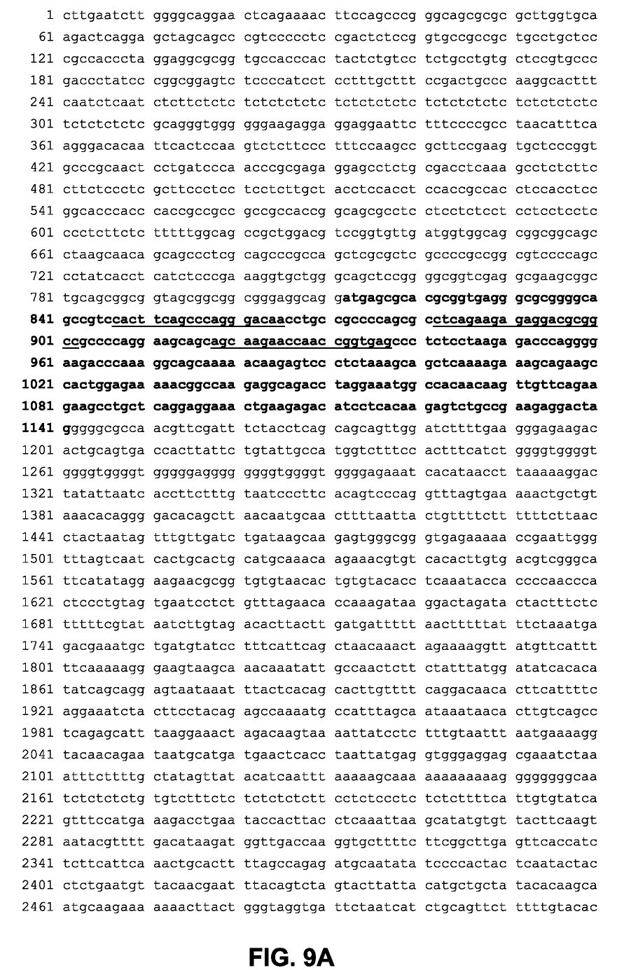

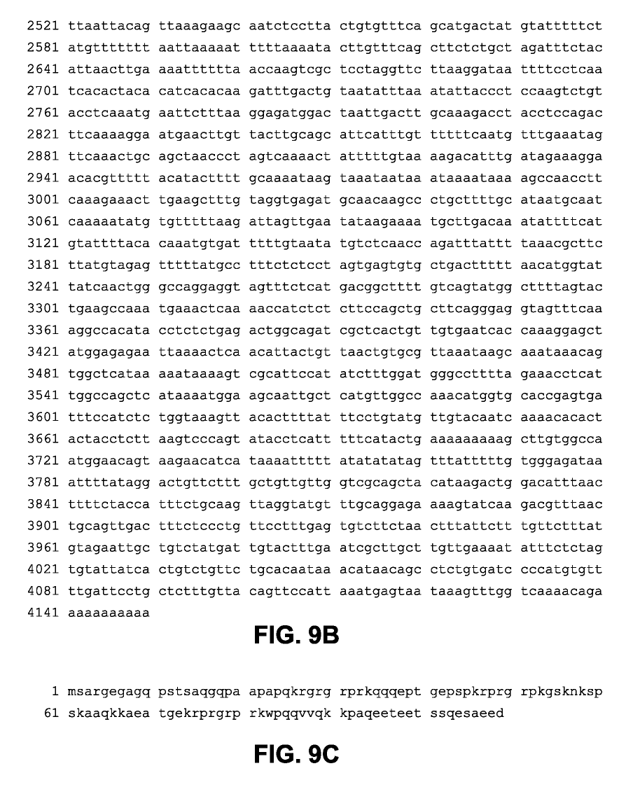

In an embodiment, the above-mentioned method comprises a step of amplification. In an embodiment, the level of expression of one or more of the genes identified herein (e.g., those listed in Tables 2A and 2B) is measured and the method comprises amplifying one or more nucleic acids using suitable primers. In an embodiment, the level of expression of HMGA2 is measured and the method comprises amplifying a HMGA2 nucleic acid using a suitable pair of primers. Suitable pairs of primers may be designed based on the nucleotide sequence of HMGA2 (FIGS. 9A and 9B, SEQ ID NO: 10). In an embodiment, each of the HMGA2 primer comprises from about 7-8 to about 100, 90, 80, 70, 60 or 50 nucleotides, in further embodiments from about 10 to about 50, 45 or 40 nucleotides, from about 10 to about 35 nucleotides, from about 10 to about 35, 34, 33, 32, 31 or 30 nucleotides, from about 15 to about 25 nucleotides or from about 16 to about 24 nucleotides. In an embodiment, each of the HMGA2 primer comprises about 10, 11, 12, 13, 14, 15, 16, 17, 18, 19, 20, 21, 22, 23, 24, 25, 26, 27, 28, 29 or 30 nucleotides. In an embodiment, each of the HMGA2 primer comprises a sequence corresponding to at least 10 nucleotides (e.g., contiguous nucleotides) of SEQ ID NO: 10, or its complement.

In an embodiment, the pair of HMGA2 primers comprises a first HMGA2 primer comprising at least 10 nucleotides of the sequence 5′-CACTTCAGCCCAGGGACAA-3′ (SEQ ID NO: 1) and/or a second HMGA2 primer comprising at least 10 nucleotides of the sequence 5′-CTCACCGGTTGGTTCTTGCT-3′ (SEQ ID NO: 2). In an embodiment, the first HMGA2 primer comprises at least 11, 12, 13, 14, 15, 16, 17, 18 or 19 nucleotides of the sequence 5′-CACTTCAGCCCAGGGACAA-3′ (SEQ ID NO: 1). In a further embodiment, the first HMGA2 primer comprises, or consists of, the sequence 5′-CACTTCAGCCCAGGGACAA-3′ (SEQ ID NO: 1). In an embodiment, the second HMGA2 primer comprises at least 11, 12, 13, 14, 15, 16, 17, 18, 19 or 20 nucleotides of the sequence 5′-CTCACCGGTTGGTTCTTGCT-3′. (SEQ ID NO: 2). In a further embodiment, the second HMGA2 primer comprises, or consists of, the sequence 5′-CTCACCGGTTGGTTCTTGCT-3′. (SEQ ID NO: 2).

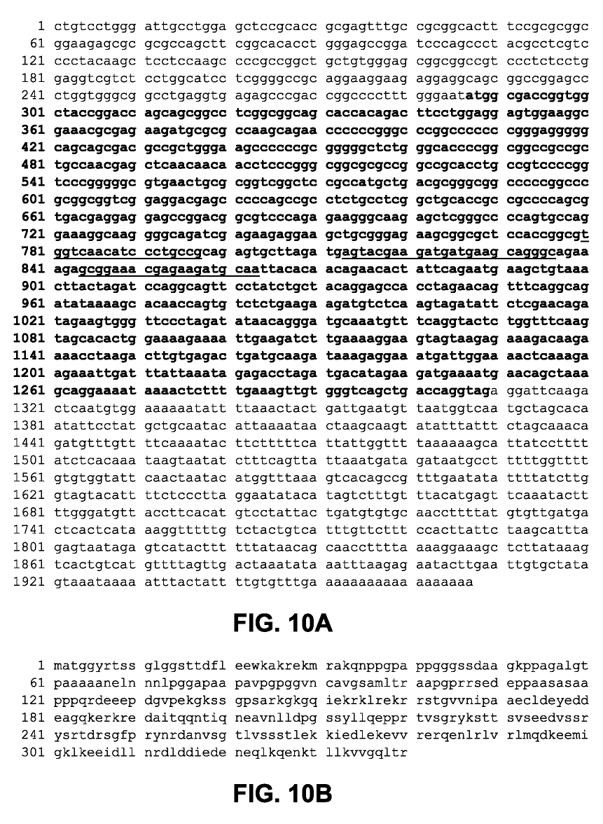

In an embodiment, the level of expression of PAWR is measured and the method comprises amplifying a PAWR nucleic acid using a suitable pair of primers. Suitable pairs of primers may be designed based on the nucleotide sequence of PAWR (FIG. 10A, SEQ ID NO: 12). In an embodiment, each of the PAWR primer comprises from about 7-8 to about 100, 90, 80, 70, 60 or 50 nucleotides, in further embodiments from about 10 to about 50, 45 or 40 nucleotides, from about 10 to about 35 nucleotides, from about 10 to about 35, 34, 33, 32, 31 or 30 nucleotides, from about 15 to about 25 nucleotides or from about 16 to about 24 nucleotides. In an embodiment, each of the PAWR primer comprises about 10, 11, 12, 13, 14, 15, 16, 17, 18, 19, 20, 21, 22, 23, 24, 25, 26, 27, 28, 29 or 30 nucleotides. In an embodiment, each of the PAWR primer comprises a sequence corresponding to at least 10 nucleotides (e.g., contiguous) of SEQ ID NO: 12), or its complement.

In an embodiment, the pair of PAWR primers comprises a first PAWR primer comprising at least 10 nucleotides of the sequence 5′-TGGTCAACATCCCTGCCG-3′ (SEQ ID NO: 7) and/or a second PAWR primer comprising at least 10 nucleotides of the sequence 5′-TTGCATCTTCTCGTTTCCGC-3′ (SEQ ID NO: 8). In an embodiment, the first PAWR primer comprises at least 11, 12, 13, 14, 15, 16, 17 or 18 nucleotides of the sequence 5′-TGGTCAACATCCCTGCCG-3′ (SEQ ID NO: 7). In a further embodiment, the first PAWR primer comprises, or consists of, the sequence 5′-TGGTCAACATCCCTGCCG-3′ (SEQ ID NO: 7). In an embodiment, the second PAWR primer comprises at least 11, 12, 13, 14, 15, 16, 17, 18, 19 or 20 nucleotides of the sequence 5′-TTGCATCTTCTCGTTTCCGC-3′ (SEQ ID NO: 8). In a further embodiment, the second PAWR primer comprises, or consists of, the sequence 5′-TTGCATCTTCTCGTTTCCGC-3′ (SEQ ID NO: 8).

The nucleic acid(s) or amplification product(s) may be detected or quantified by hybridizing a probe (e.g., a labeled probe) to a portion of the nucleic acid(s) or amplification product(s). The probe may be labelled with a detectable group that may be, for example, a fluorescent moiety, chemiluminescent moiety, radioisotope, biotin, avidin, enzyme, enzyme substrate, or other reactive group. Other well-known detection techniques include, for example, gel filtration, gel electrophoresis and visualization of the amplicons, and High Performance Liquid Chromatography (HPLC). In certain embodiments, for example using real-time TMA or real-time PCR, the level of amplified product is detected as the product accumulates.

In an embodiment, the above-mentioned method comprises a step of detection or quantification with one or more probes. In an embodiment, the level of expression of one or more of the genes identified herein (e.g., those listed in Tables 2A and 2B) is measured and the method comprises detecting or quantifying the one or more nucleic acids or amplified products with a specific probes. In an embodiment, the level of expression of HMGA2 is measured and the method comprises detecting or quantifying the HMGA2 nucleic acid or amplified product with a HMGA2 probe. Suitable HMGA2 probes may be designed based on the nucleotide sequence of HMGA2 (FIGS. 9A and 9B, SEQ ID NO: 10) In an embodiment, the HMGA2 probe comprises from about 7-8 to about 100, 90, 80, 70, 60 or 50 nucleotides, in further embodiments from about 10 to about 50, 45 or 40 nucleotides, from about 10 to about 35 nucleotides, from about 10 to about 35, 34, 33, 32, 31 or 30 nucleotides, from about 15 to about 25 nucleotides or from about 16 to about 24 nucleotides. In an embodiment, each of the primer comprises about 10, 11, 12, 13, 14, 15, 16, 17, 18, 19, 20, 21, 22, 23, 24, 25, 26, 27, 28, 29 or 30 nucleotides. In an embodiment, the HMGA2 probe comprises a sequence corresponding to at least 10 nucleotides (e.g., contiguous) of SEQ ID NO: 10, or its complement.

In an embodiment, the HMGA2 probe comprises at least about 10 nucleotides of the sequence 5′-CTCAGAAGAGAGGACGCGGCC-3′ (SEQ ID NO: 3). In an embodiment, the HMGA2 probe comprises at least about 11, 12, 13, 14, 15, 16, 17, 18, 19, 20 or 21 nucleotides of the sequence 5′-CTCAGAAGAGAGGACGCGGCC-3′ (SEQ ID NO: 3). In an embodiment, the HMGA2 probe consists of the sequence 5′-CTCAGAAGAGAGGACGCGGCC-3′ (SEQ ID NO: 3).

In an embodiment, the level of expression of PAWR is measured and the method comprises detecting or quantifying the PAWR nucleic acid or amplified product with a PAWR probe. Suitable PAWR probes may be designed based on the nucleotide sequence of PAWR (FIG. 10A, SEQ ID NO: 12) In an embodiment, the PAWR probe comprises from about 7-8 to about 100, 90, 80, 70, 60 or 50 nucleotides, in further embodiments from about 10 to about 50, 45 or 40 nucleotides, from about 10 to about 35 nucleotides, from about 10 to about 35, 34, 33, 32, 31 or 30 nucleotides, from about 15 to about 25 nucleotides or from about 16 to about 24 nucleotides. In an embodiment, the PAWR probe comprises about 10, 11, 12, 13, 14, 15, 16, 17, 18, 19, 20, 21, 22, 23, 24, 25, 26, 27, 28, 29 or 30 nucleotides. In an embodiment, the PAWR probe comprises a sequence corresponding to at least 10 nucleotides (e.g., contiguous) of SEQ ID NO: 12, or its complement.

In an embodiment, the PAWR probe comprises at least about 10 nucleotides of the sequence 5′-AGTACGAAGATGATGAAGCAGGGC-3′ (SEQ ID NO: 9). In an embodiment, the PAWR probe comprises at least about 11, 12, 13, 14, 15, 16, 17, 18, 19, 20, 21, 22, 23 or 24 nucleotides of the sequence 5′-AGTACGAAGATGATGAAGCAGGGC-3′ (SEQ ID NO: 9). In an embodiment, the PAWR probe comprises, or consists of, the sequence 5′-AGTACGAAGATGATGAAGCAGGGC-3′ (SEQ ID NO: 9).

In an embodiment, the above-mentioned method comprises a step of normalizing the gene expression levels, i.e. normalization of the measured levels of the above-noted genes against a stably expressed control gene (or housekeeping gene) to facilitate the comparison between different samples. “Normalizing” or “normalization” as used herein refers to the correction of raw gene expression values/data between different samples for sample to sample variations, to take into account differences in “extrinsic” parameters such as cellular input, nucleic acid (RNA) or protein quality, efficiency of reverse transcription (RT), amplification, labeling, purification, etc., i.e. differences not due to actual “intrinsic” variations in gene expression by the cells in the samples. Such normalization is performed by correcting the raw gene expression values/data for a test gene (or gene of interest) based on the gene expression values/data measured for one or more “housekeeping” or “control” genes, i.e. whose expressions are known to be constant (i.e. to show relatively low variability) between the cells of different tissues and under different experimental conditions. Thus, in an embodiment, the above-mentioned method further comprises measuring the level of expression of a housekeeping gene in the biological sample. Suitable housekeeping genes are known in the art and several examples are described in WO 2014/134728, including those listed in Table 1.

TABLE 1
Examples of housekeeping genes

| Housekeeping | |
|---|---|
| Gene | GenBank Accession number |
| ABI1 | NM_001012750, NM_001012751, NM_001012752, |
| | NM_001178116, NM_001178119, NM_001178120, |
| | NM_001178121, NM_001178122, NM_001178123, |
| | NM_001178124, NM_001178125, NM_005470 |
| ACIN1 | NM_001164814, NM_001164815, NM_001164816, |
| | NM_001164817, NM_014977 |
| ACP1 | NM_001040649, NM_004300, NM_007099 |
| ADAR | NM_001025107, NM_001111, NM_001193495 |
| ADD1 | NM_001119, NM_014189, NM_014190, NM_176801 |
| ANAPC5 | NM_001137559, NM_016237 |
| ARF1 | NM_001024226, NM_001024227, NM_001024228, |
| | NM_001658 |
| ATP5B | NM_001686 |
| ATP6V1G1 | NM_004888 |
| ATXN2L | NM_007245, NM_017492, NM_145714, |
| | NM_148414, NM_148415, NM_148416 |
| AUP1 | NM_181575 |
| C1orf144 | NM_001114600, NM_015609 |
| C20orf43 | NM_016407 |
| C6orf62 | NM_030939 |
| CAPRIN1 | NM_005898, NM_203364 |
| CASC3 | NM_007359 |
| CCNI | NM_006835 |
| CDC37 | NM_007065 |
| CDV3 | NM_001134422, NM_001134423, NM_017548 |
| CMPK1 | NM_001136140, NM_016308 |
| CMTM3 | NM_144601, NM_181553 |
| COPB1 | NM_001144061, NM_001144062, NM_016451 |
| COPS5 | NM_006837 |
| CS | NM_004077 |
| CSDE1 | NM_001007553, NM_001130523, NM_001242891, |
| | NM_001242892, NM_001242893, NM_007158 |
| DAP3 | NM_001199849, NM_001199850, NM_001199851, |
| | NM_004632, NM_033657 |
| DCAF8 | NM_015726 |
| DDX5 | NM_004396 |
| DLST | NM_001933 |
| DNAJC7 | NM_001144766, NM_003315 |
| DOCK2 | NM_004946 |
| E2F4 | NM_001950 |
| EIF3I | NM_003757 |
| EIF4H | NM_022170, NM_031992 |
| EWSR1 | NM_001163285, NM_001163286, NM_001163287, |
| | NM_005243, NM_013986 |
| FAM32A | NM_014077 |
| GABARAPL2 | NM_007285 |
| GNB1 | NM_002074, NM_001282538, NM_001282539 |
| GORASP2 | NM_001201428, NM_015530 |
| GTF2F1 | NM_002096 |
| HDAC3 | NM_003883 |
| HNRNPA2B1 | NM_002137, NM_031243 |
| HNRNPC | NM_001077442, NM_001077443, NM_004500, |
| | NM_031314 |
| HNRNPD | NM_002138, NM_031369, NM_031370 |
| HNRNPH3 | NM_012207, NM_021644 |
| HNRNPK | NM_002140, NM_031262, NM_031263 |
| HNRNPL | NM_001005335, NM_001533 |
| HNRNPU | NM_004501, NM_031844 |
| HNRNPUL1 | NM_007040 |
| IDH3B | NM_001258384, NM_006899, NM_174855, |
| | NM_174856 |
| IK | NM_006083 |
| KARS | NM_001130089, NM_005548 |
| KHDRBS1 | NM_006559 |
| LSM14A | NM_001114093, NM_015578 |
| MAPRE1 | NM_012325 |
| MARS | NM_004990 |
| MLF2 | NM_005439 |
| MMADHC | NM_015702 |
| MORF4L1 | NM_001265605, NM_006791, NM_206839 |
| MRFAP1 | NM_033296 |
| MRPL9 | NM_031420 |
| MTA2 | NM_004739 |
| MYL12B | NM_001144944, NM_001144945, NM_033546 |
| NOL7 | NM_016167 |
| NRD1 | NM_001101662, NM_001242361, NM_002525 |
| OCIAD1 | NM_001079839, NM_001079840, NM_001079841, |
| | NM_001079842, NM_001168254, NM_017830 |
| PAPOLA | NM_001252006, NM_032632 |
| PCBP2 | NM_001098620, NM_001128911, NM_001128912, |
| | NM_001128913, NM_001128914, NM_005016, |
| | NM_031989 |
| POLR2C | NM_032940 |
| PSMA1 | NM_002786, NM_148976 |
| PSMB1 | NM_002793 |
| PSMD2 | NM_002808 |
| PSMD6 | NM_014814, NM_001271780, NM_001271779, |
| | NM_001271781 |
| PSMD7 | NM_002811 |
| PSME1 | NM_006263, NM_176783 |
| PSME3 | NM_001267045, NM_005789, NM_176863 |
| PSMF1 | NM_006814, NM_178578 |
| PTPRA | NM_002836, NM_080840, NM_080841 |
| RAB7A | NM_004637 |
| RBM22 | NM_018047 |
| RBM8A | NM_005105 |
| RHOA | NM_001664 |
| RNF114 | NM_018683 |
| RNF7 | NM_001201370, NM_014245, NM_183237 |
| SEC22B | NM_0048925 |
| SEC31A | NM_001077206, NM_001077207, NM_001077208, |
| | NM_001191049, NM_014933, NM_016211 |
| SERP1 | NM_014445 |
| SF3A1 | NM_001005409, NM_005877 |
| SF3B2 | NM_006842 |
| SLC25A3 | NM_002635, NM_005888, NM_213611 |
| SNW1 | NM_012245 |
| SON | NM_032195, NM_138927 |
| SRP14 | NM_003134 |
| SRPR | NM_001177842, NM_003139 |
| SRSF5 | NM_001039465, NM_006925 |
| SRSF9 | NM_003769 |
| SSR2 | NM_003145 |
| STX16 | NM_001001433, NM_001134772, NM_001134773, |
| | NM_001204868, NM_003763 |
| SUMO1 | NM_001005781, NM_001005782, NM_003352 |
| SUMO3 | NM_006936 |
| SUPT6H | NM_003170 |
| TCEB1 | NM_001204857, NM_001204858, NM_001204859, |
| | NM_001204860, NM_001204861, NM_001204862, |
| | NM_001204863, NM_001204864, NM_005648 |
| TH1L | NM_198976 |
| TMED2 | NM_006815 |
| TMEM50A | NM_014313 |
| TRIP12 | NM_004238 |
| U2AF1 | NM_001025203, NM_001025204, NM_006758 |
| UBE2D3 | NM_003340, NM_181886, NM_181887, |
| | NM_181888, NM_181889, NM_181890, |
| | NM_181891, NM_181892, NM_181893 |
| UBE2I | NM_003345, NM_194259, NM_194260, NM_194261 |
| UBE2Z | NM_023079 |
| UBQLN1 | NM_013438, NM_053067 |
| USP39 | NM_001256725, NM_001256726, NM_001256728, |
| | NM_006590 |
| USP4 | NM_001251877, NM_003363, NM_199443 |
| VCP | NM_007126 |
| VPS4A | NM_013245 |
| XRN2 | NM_012255 |
| YME1L1 | NM_014263, NM_139312 |
| ZC3H11A | NM_001271675, NM_014827 |
| ZNF207 | NM_001032293, NM_001098507, NM_003457 |

Other commonly used housekeeping genes include TBP, YWHAZ, PGK1, LDHA, ALDOA, HPRT1, SDHA, UBC, GAPDH, ACTB, G6PD, VIM, TUBA1A, PFKP, B2M, GUSB, PGAM1, HMBS.

In a further embodiment, the method further comprises measuring the level of expression of one or more housekeeping genes in a biological sample from the subject/patient. In an embodiment, the level of expression of the housekeeping gene is measured and the method comprises amplifying a housekeeping gene nucleic acid using a suitable pair of primers. In an embodiment, the housekeeping gene used for normalization is ABL1. In another embodiment, the housekeeping gene used for normalization is PSMA1. In an embodiment, the method comprises amplifying an ABL1 nucleic acid using a suitable pair of primers. Suitable pairs of primers may be designed based on the nucleotide sequence of ABL1, which may be found in GenBank Accession No. NM_001012750, NM_001012751, NM_001012752, NM_001178116, NM_001178119, NM_001178120, NM_001178121, NM_001178122, NM_001178123, NM_001178124, NM_001178125 and NM_005470. In an embodiment, the pair of primer comprises a first primer comprising at least 10 nucleotides of the sequence 5′-TGGAGATAACACTCTAAGCATAACTAAAGGT-3′ (SEQ ID NO: 4) and/or a second primer comprising at least 10 nucleotides of the sequence 5′-GATGTAGTTGCTTGGGACCCA-3′ (SEQ ID NO: 5). In an embodiment, the first primer comprises at least 11, 12, 13, 14, 15, 16, 17, 18, 19, 20, 21, 22, 23, 24, 25, 26, 27, 28, 29, 30 or 31 nucleotides of the sequence 5′-TGGAGATAACACTCTAAGCATAACTAAAGGT-3′ (SEQ ID NO: 4). In a further embodiment, the first primer comprises, or consists of the sequence 5′-TGGAGATAACACTCTAAGCATAACTAAAGGT-3′ (SEQ ID NO: 4). In an embodiment, the second primer comprises at least 11, 12, 13, 14, 15, 16, 17, 18, 19, 20 or 21 nucleotides of the sequence 5′-GATGTAGTTGCTTGGGACCCA-3′ (SEQ ID NO: 5). In a further embodiment, the second primer comprises, or consists of, the sequence 5′-GATGTAGTTGCTTGGGACCCA-3′ (SEQ ID NO: 5).

In an embodiment, the above-mentioned method comprises a step of detection or quantification of the housekeeping gene nucleic acid (e.g. ABL1) with a probe. In an embodiment, the housekeeping gene is ABL1 and the probe comprises at least 10 nucleotides of the sequence 5′-CCATTTTTGGTTTGGGCTTCACACCATT-3′ (SEQ ID NO: 6). In an embodiment, the probe comprises at least 11, 12, 13, 14, 15, 16, 17, 18, 19, 20, 21, 22, 23, 24, 25, 26, 27 or 28 nucleotides of the sequence 5′-CCATTTTTGGTTTGGGCTTCACACCATT-3′ (SEQ ID NO: 6). In a further embodiment, the probe comprises, or consists of, the sequence 5′-CCATTTTTGGTTTGGGCTTCACACCATT-3′ (SEQ ID NO: 6).

In an embodiment, one or more of the primers and/or probes is/are detectably labelled, i.e. comprises a detectable label attached thereto. As used herein, the term “detectable label” refers to a moiety emitting a signal (e.g., light) that may be detected using an appropriate detection system. Any suitable detectable label may be used in the method described herein. Detectable labels include, for example, enzyme or enzyme substrates, reactive groups, chromophores such as dyes or colored particles, luminescent moieties including bioluminescent, phosphorescent or chemiluminescent moieties, and fluorescent moieties. In an embodiment, the detectable label is a fluorescent moiety. Fluorophores that are commonly used include, but are not limited to, fluorescein, 5-carboxyfluorescein (FAM), 2′7′-dimethoxy-4′5′-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′-dimethylaminophenylazo) benzoic acid (DABCYL), and 5-(2′-aminoethyl)aminonaphthalene-1-sulfonic acid (EDANS). The fluorophore may be any fluorophore known in the art, including, but not limited to: FAM, TET, HEX, Cy3, TMR, ROX, Texas Red®, LC red 640, Cy5, and LC red 705. Fluorophores for use in the methods and compositions provided herein may be obtained commercially, for example, from Biosearch Technologies (Novato, Calif.), Life Technologies (Carlsbad, Calif.), GE Healthcare (Piscataway N.J.), Integrated DNA Technologies (Coralville, Iowa) and Roche Applied Science (Indianapolis, Ind.). In some embodiments, the fluorophore is chosen to be usable with a specific detector, such as a specific spectrophotometric thermal cycler, depending on the light source of the instrument. In some embodiments, if the assay is designed for the detection of two or more target nucleic acids (e.g., multiplex assays, duplex PCR), for example HMGA2 and one or more additional prognostic markers such as PAWR, two or more different fluorophores may be chosen with absorption and emission wavelengths that are well separated from each other (i.e., have minimal spectral overlap). In some embodiments, the fluorophore is chosen to work well with one or more specific quenchers. A representative example of a suitable combination of fluorescent label and quenchers is FAM/ZEN/IBFQ, which comprises the fluorescent FAM (excitation max.=494 nm, emission max.=520 nm), the ZEN™ quencher (non-abbreviation; absorption max 532 nm), and the Iowa black fluorescein quencher (IBFQ, absorption max=531 nm) (Integrated DNA Technologies®). Covalent attachment of detectable label and/or quencher to primer and/or probe can be accomplished according to standard methodology well known in the art as discussed, for example in Sambrook et al. (Molecular Cloning: A Laboratory Manual, Cold Spring Harbor, N.Y., 2001), Ausubel et al. (In Current Protocols in Molecular Biology, John Wiley & Sons, New York, 1998), Eckstein, editor, Oligonucleotides and Analogues: A Practical Approach (IRL Press, Oxford, 1991); Zuckerman et al., Nucleic Acids Research, 15: 5305-5321 (1987) (3′ thiol group on oligonucleotide); Sharma et al., Nucleic Acids Research, 19:3019 (1991) (3′ sulfhydryl); Giusti et al., PCR Methods and Applications, 2:223-227 (1993) and Fung et al, U.S. Pat. No. 4,757,141 (5′ phosphoamino group via Aminolink™ II available from Applied Biosystems®, Foster City, Calif.); Stabinsky, U.S. Pat. No. 4,739,044 (3′ aminoalkylphosphoryl group); Agrawal et al., Tetrahedron Letters, 31:1543-1546 (1990) (attachment via phosphoramidate linkages); Sproat et al., Nucleic Acids Research, 15:4837 (1987) (5′ mercapto group); Nelson et al., Nucleic Acids Research, 17:7187-7194 (1989) (3′ amino group); and the like.

In another embodiment, the expression of the one or more genes or encoded gene products is measured at the protein level. Methods to measure the amount/level of proteins are well known in the art. Protein levels may be detected directly using a ligand binding specifically to the protein, such as an antibody or a fragment thereof. In embodiments, such a binding molecule or reagent (e.g., antibody) is labeled/conjugated, e.g., radio-labeled, chromophore-labeled, fluorophore-labeled, or enzyme-labeled to facilitate detection and quantification of the complex (direct detection). Alternatively, protein levels may be detected indirectly, using a binding molecule or reagent, followed by the detection of the [protein/binding molecule or reagent] complex using a second ligand (or second binding molecule) specifically recognizing the binding molecule or reagent (indirect detection). Such a second ligand may be radio-labeled, chromophore-labeled, fluorophore-labeled, or enzyme-labeled to facilitate detection and quantification of the complex. Enzymes used for labeling antibodies for immunoassays are known in the art, and the most widely used are horseradish peroxidase (HRP) and alkaline phosphatase (AP). Examples of binding molecules or reagents include antibodies (monoclonal or polyclonal), natural or synthetic ligands, and the like.

Examples of methods to measure the amount/level of protein in a sample include, but are not limited to: Western blot, immunoblot, enzyme-linked immunosorbent assay (ELISA), “sandwich” immunoassays, radioimmunoassay (RIA), immunoprecipitation, surface plasmon resonance (SPR), chemiluminescence, fluorescent polarization, phosphorescence, immunohistochemical (IHC) analysis, matrix-assisted laser desorption/ionization time-of-flight (MALDI-TOF) mass spectrometry, microcytometry, microarray, antibody array, microscopy (e.g., electron microscopy), flow cytometry, proteomic-based assays, and assays based on a property or activity of the protein including but not limited to ligand binding or interaction with other protein partners, enzymatic activity, fluorescence. For example, if the protein of interest is a kinase known to phosphorylate of given target, the level or activity of the protein of interest may be determined by measuring the level of phosphorylation of the target in the presence of the test compound. If the protein of interest is a transcription factor known to induce the expression of one or more given target gene(s), the level or activity of the protein of interest may be determined by the measuring the level of expression of the target gene(s).

In an embodiment, the reference or control level of expression is a level measured in a non-cancerous (non-AML) cell sample (e.g., normal hematopoietic stem cell sample, normal CD34⁺ cell sample, etc.) or one or more AML samples (one or more AML samples from a mixture of AML subtypes/subgroups). In an embodiment, the reference or control level of expression is a level measured in a TP53 wild-type (non-mutated) sample.

“Control level” or “reference level” or “standard level” are used interchangeably herein and broadly refers to a separate baseline level measured in one or more comparable “control” samples, which may be from subjects or patients not suffering from the disease or from AML samples from different AML subtypes. The corresponding control level may be a level corresponding to an average/mean or median level calculated based of the levels measured in several reference or control subjects (e.g., a pre-determined or established standard level). The control level may be a pre-determined “cut-off” value recognized in the art or established based on levels measured in samples from one or a group of control subjects. For example, the “threshold reference level” may be a level corresponding to the minimal level of HMGA2 (and/or PAWR) expression (cut-off) that permits to distinguish in a statistically significant manner AML patients having a poor disease prognosis from those not having a poor prognosis, which may be determined using samples from AML patients with different disease outcomes, for example. Alternatively, the “threshold reference level” may be a level corresponding to the level of HMGA2 (and/or PAWR) expression (cut-off) that permits to best or optimally distinguish in a statistically significant manner AML patients having a poor disease prognosis from those not having a poor prognosis. The corresponding reference/control level may be adjusted or normalized for age, gender, race, or other parameters. The “control level” can thus be a single number/value, equally applicable to every patient individually, or the control level can vary, according to specific subpopulations of patients. Thus, for example, older men might have a different control level than younger men, and women might have a different control level than men. The predetermined standard level can be arranged, for example, where a tested population is divided equally (or unequally) into groups, such as a low-risk group, a medium-risk group and a high-risk group or into quadrants or quintiles, the lowest quadrant or quintile being individuals with the lowest risk (i.e., lowest level of expression of HMGA2 (and PAWR)) and the highest quadrant or quintile being individuals with the highest risk (i.e., highest level of expression of HMGA2 (and PAWR)). It will also be understood that the control levels according to the invention may be, in addition to predetermined levels or standards, levels measured in other samples (e.g. from healthy/normal subjects, or AML patients) tested in parallel with the experimental sample. The reference or control levels may correspond to normalized levels, i.e. reference or control values subjected to normalization based on the expression of a housekeeping gene. In an embodiment, the threshold reference level corresponds to a normalized value of HMGA2 (and PAWR) copy number of at least about 700 to 1300, for example about 700, 750, 800, 850, 900, 950, 1000, 1050, 1100, 1150, 1200, 1250 or 1300 per 10⁴ ABL1 copy number, as described herein. In an embodiment, the threshold reference level corresponds to a normalized value of HMGA2 copy number of about 1050 per 10⁴ ABL1 copy number, as described herein. In an embodiment, the threshold reference level corresponds to a normalized value of HMGA2 copy number of about 1100 per 10⁴ ABL1 copy number, as described herein. In an embodiment, the threshold reference level corresponds to a normalized value of PAWR copy number of about 900 per 10⁴ ABL1 copy number, as described herein. In an embodiment, the threshold reference level corresponds to a normalized value of PAWR copy number of about 950 per 10⁴ ABL1 copy number, as described herein. The skilled person would understand that a corresponding threshold reference level, which would define a similar threshold value for HMGA2 (and PAWR) expression levels, may be calculated based, for example, on the expression of another housekeeping gene or using another method of calculation.

“Higher expression” or “higher level of expression” as used herein refers to (i) higher expression of the one or more genes, for example HMGA2 (and PAWR) (protein and/or mRNA) in one or more given cells present in the sample (relative to the control) and/or (ii) higher amount of cells expressing the one or more genes, for example HMGA2 (and PAWR) in the sample (relative to the control). In an embodiment, higher refers to a level of expression that is above the control level (e.g., the predetermined cut-off value). In another embodiment, higher refers to a level of expression that is at least one standard deviation above the control level (e.g., the predetermined cut-off value) (e.g. that is statistically significant as determined using a suitable statistical analysis). In other embodiments, higher refers to a level of expression that is at least 1.5, 2, 2.5, 3, 4 or 5 standard deviations above the control level (e.g., the predetermined cut-off value. In another embodiment, “higher expression” refers to an expression that is at least 10, 20, 30, 40 or 50% higher in the test sample relative to the control level. In another embodiment, higher refers to a level of expression that is at least 1.5, 2-, 5-, 10-, 25-, or 50-fold higher in the test sample relative to the control level (e.g., the predetermined cut-off value).

“Lower expression” or “lower level of expression” as used herein refers to (i) lower expression of the one or more genes (protein and/or mRNA) in one or more given cells present in the sample (relative to the control) and/or (ii) lower amount of cells expressing the one or more genes in the sample (relative to the control). In an embodiment, lower refers to a level of expression that is below the control level (e.g., the predetermined cut-off value). In another embodiment, lower refers to a level of expression that is at least one standard deviation below the control level (e.g., the predetermined cut-off value) (e.g. that is statistically significant as determined using a suitable statistical analysis). In other embodiments, lower refers to a level of expression that is at least 1.5, 2, 2.5, 3, 4 or 5 standard deviations below the control level (e.g., the predetermined cut-off value. In another embodiment, “lower expression” refers to an expression that is at least 10, 20, 30, 40 or 50% lower in the test sample relative to the control level. In another embodiment, lower refers to a level of expression that is at least 1.5, 2-, 5-, 10-, 25-, or 50-fold lower in the test sample relative to the control level (e.g., the predetermined cut-off value).

In another embodiment, the method described herein further comprises obtaining or collecting a biological sample comprising leukemic cells from a subject/patient. In various embodiments, the sample can be from any source that contains biological material suitable for the detection of the nucleic acid(s), such as genomic DNA, RNA (cDNA), and/or proteins, for example a tissue or cell sample from the subject/patient (blood cells, bone marrow cells, etc. that comprises leukemic cells (AML cells). The sample may be subjected to cell purification/enrichment techniques to obtain a cell population enriched in a specific cell subpopulation or cell type(s). The sample may be subjected to commonly used isolation and/or purification techniques for enrichment in nucleic acids (genomic DNA, cDNA, mRNA) and/or proteins. Accordingly, in an embodiment, the method may be performed on an isolated nucleic acid and/or protein sample, such as cDNA. The biological sample may be collected using any methods for collection of biological fluid, tissue or cell sample, such as venous puncture for collection of blood cell samples. Thus, the term “biological sample comprising leukemic cells” as used herein refers to a crude leukemic cell sample, a sample enriched in certain cells (i.e., that has been subjected to cell purification/enrichment techniques), or isolated nucleic acids (RNA, cDNA) and/or proteins from leukemic cells (subjected or not to nucleic acid amplification). In an embodiment, the biological sample comprising leukemic cells comprises nucleic acids (RNA, cDNA) obtained or isolated from leukemic cells.

In certain embodiments, methods of prognosis/diagnosis described herein may be at least partly, or wholly, performed in vitro. In a further embodiment, the method is wholly performed in vitro.

In another aspect, the present invention provides a method of detecting a HMGA2 nucleic acid in a patient suffering from AML (e.g., an intermediate risk AML patient), said method comprising: obtaining a biological sample comprising leukemic cells from said patient; and detecting the level of the HMGA2 nucleic acid in the sample by contacting the sample with one or more oligonucleotides specific for HMGA2 and detecting hybridization between the HMGA2 nucleic acid and the one or more oligonucleotides.

In an embodiment, the above-mentioned method further comprises selecting and/or administering a course of therapy to said subject/patient in accordance with the diagnostic/prognostic result. For example, if it is determined that the subject/patient has a poor disease prognosis, a more aggressive or a treatment regimen adapted for treatment of poor prognosis AML may be used, such as for example a more aggressive or non-standard chemotherapy regimen and/or stem cell/bone marrow transplantation (e.g., allogeneic transplantation). The method further comprises subjecting the subject/patient to a suitable anti-leukemia therapy (e.g., bone marrow or hematopoietic stem cell transplantation, chemotherapy, etc.) in accordance with the prognostic result. For example, for good-prognosis leukemia, the therapy comprises an induction chemotherapy with agents such as cytarabine (ara-C) and an anthracycline (e.g., daunorubicin), followed by a consolidation therapy comprising three, four or five courses of intensive chemotherapy. For poor-prognosis leukemia, the consolidation therapy typically comprises stem cell/bone marrow transplantation, e.g., allogeneic stem cell transplantation.

In another aspect, the present invention provides a method for treating an AML patient having a good or poor disease prognosis identified using the method described herein, said method comprising treating said patient with a suitable treatment regimen for good or poor prognosis AML.

In another aspect, the present invention provides a method for treating an AML patient having a poor disease prognosis identified using the method described herein, said method comprising treating said patient with a suitable treatment regimen for poor prognosis AML, including an experimental treatment regimen.

In an embodiment, the above-mentioned method for treating further comprises a step of identifying an AML patient having a good or poor disease prognosis using the methods described herein.

In another aspect, the present invention provides a method for treating an AML patient having a good or poor disease prognosis comprising (i) identifying an AML patient having a poor disease prognosis using the methods described herein; and (ii) treating said patient with a suitable treatment regimen for good or poor prognosis AML.

In another aspect, the present invention provides a method for treating an AML patient having a poor disease prognosis comprising (i) identifying an AML patient having a good or poor disease prognosis using the methods described herein; and (ii) treating said patient with a suitable treatment regimen for poor prognosis AML.

In another aspect, the present invention provides an assay mixture for the assessment of AML (e.g., for the prognosis of AML), the assay mixture comprising: (i) a biological sample from a patient suffering from AML; and (ii) one or more reagents for determining/measuring the level of expression of HMGA2 in the sample.

In another aspect, the present invention provides a system for the assessment of AML (e.g., for the prognosis of AML), comprising: a biological sample obtained from an AML patient; and one or more assays to determine the level of expression of one or more of the markers/genes described herein (e.g., HMGA2 and/or PAWR).

The present invention provides a system for the assessment of AML (e.g., for the prognosis of AML) in a patient in need thereof, comprising: a sample analyzer configured to produce a signal for one or more of the markers/genes described herein (e.g., HMGA2 and/or PAWR) in a biological sample of the patient; and a computer sub-system programmed to calculate, based on the one or more of the markers/genes, whether the signal is higher or lower than a reference value. In various embodiments, the system further comprises the biological sample.

In another aspect, the present invention provides an assay mixture for the assessment of AML (e.g., for the diagnosis of TP53mut AML), the assay mixture comprising: (i) a biological sample from a patient suffering from AML; and (ii) one or more reagents for determining/measuring the level of expression of one or more of the genes of Tables 2A and/or 2B in the sample.

In another aspect, the present invention provides a system for the assessment of AML (e.g., for the diagnosis of TP53mut AML), comprising: a biological sample obtained from an AML patient; and one or more assays to determine the level of expression of one or more of the markers/genes described herein (e.g., those listed in Tables 2A and/or 2B).

The present invention provides a system for the assessment of AML (e.g., for the diagnosis of TP53mut AML) in a subject in need thereof, comprising: a sample analyzer configured to produce a signal for one or more of the markers/genes of Tables 2A and/or 2B in a biological sample of the subject; and a computer sub-system programmed to calculate, based on the one or more of the markers/genes, whether the signal is higher or lower than a reference value. In various embodiments, the system further comprises the biological sample.

In another aspect, the present invention provides a kit for the assessment of AML (e.g., for the prognosis of AML), the kit comprising: (i) one or more reagents for determining/measuring the level of expression of HMGA2 in a biological sample.

In another aspect, the present invention provides a kit for the assessment of AML (e.g., for the diagnosis of TP53mut AML), the kit comprising: (i) one or more reagents for determining/measuring the level of expression of one or more of the genes of Tables 2A and/or 2B in the sample.

In an embodiment, the assay mixture or kit further comprises reagents for determining/measuring the level of expression of detecting at least 1, 2, 3, 4, or 5 additional prognostic markers in the biological sample. In a further embodiment, the assay mixture further comprises reagents for determining/measuring the level of expression of PAWR in the biological sample.

In an embodiment, the one or more reagents comprise, for example, primer(s), probe(s), antibody(ies), solution(s), buffer(s), nucleic acid amplification reagent(s) (e.g., DNA polymerase, DNA polymerase cofactor, dNTPs), nucleic acid hybridization/detection reagent(s), and/or reagents for detecting antigen-antibody complexes, etc. In an embodiment, the assay mixture or kit comprises one or more pairs of primers for amplifying a HMGA2 nucleic acid. In an embodiment, the assay mixture or kit further comprises one or more pairs of primers for amplifying a PAWR nucleic acid. In an embodiment, the assay mixture or kit comprises one or more probes for detecting one or more nucleic acids correspond to one or more additional prognostic markers. In an embodiment, the assay mixture or kit further comprises one or more reagents for determining/measuring the level of expression of at least one normalization/housekeeping gene (e.g., ABL1) in the sample. Examples of suitable pair of primers for amplifying a ABL1 nucleic acid, and of suitable probes for detecting a ABL1 nucleic acid, are described above.

In an embodiment, the assay mixture or kit for the prognosis of AML further comprises (i) a pair of primers suitable for amplifying a PAWR nucleic acid in the sample. In an embodiment, the assay mixture or kit for the prognosis of AML comprises (i) a probe suitable for detecting a PAWR nucleic acid in the sample. In another embodiment, the assay mixture or kit for the prognosis of AML comprises (i) a pair of primers suitable for amplifying a PAWR nucleic acid in the sample; and (ii) a probe suitable for detecting a PAWR nucleic acid in the sample. Examples of suitable pair of primers for amplifying a PAWR nucleic acid, and of suitable probes for detecting a PAWR nucleic acid, are described above. In an embodiment, the assay mixture or kit further comprises one or more reagents (e.g., primers and/or probes) for determining/measuring the level of expression of one or more AML prognostic markers in the sample. In an embodiment, the assay mixture or kit comprises reagents (e.g., primers and/or probes) for determining/measuring the level of expression of at least two AML prognostic markers in the sample.

Furthermore, in an embodiment, the kit may be divided into separate packages or compartments (e.g., vials, bottles) containing the respective reagent components explained above.

In addition, such a kit may optionally comprise one or more of the following: (1) instructions for using the reagents for the diagnosis and/or prognosis of AML according to the methods described herein; (2) one or more containers; and/or (3) appropriate controls/standards. Such a kit can include reagents for collecting a biological sample from a patient and reagents for processing the biological sample. The kits featured herein can also include an instruction sheet describing how to perform the assays for measuring gene expression. The instruction sheet can also include instructions for how to determine a reference cohort (control patient population), including how to determine expression levels of genes in the reference cohort and how to assemble the expression data to establish a reference for comparison to a test patient. The instruction sheet can also include instructions for assaying gene expression in a test patient and for comparing the expression level with the expression in the reference cohort to subsequently determine the appropriate treatment regimen for the test patient.

Informational material included in the kits can be descriptive, instructional, marketing or other material that relates to the methods described herein and/or the use of the reagents for the methods described herein. For example, the informational material of the kit can contain contact information, e.g., a physical address, email address, website, or telephone number, where a user of the kit can obtain substantive information about performing a gene expression analysis and interpreting the results, particularly as they apply to an AML patient's likelihood of having a poor prognosis/outcome.

The kits featured herein can also contain software necessary to infer a patient's likelihood of having a poor prognosis/outcome from the gene expression data.

In another aspect, there is provided the use of the kit, system or assay mixture described herein for prognosis of a patient suffering from AML.

### MODE(S) FOR CARRYING OUT THE INVENTION

The present invention is illustrated in further details by the following non-limiting examples.

### Example 1: Materials and Methods

Specimen Collection.

All leukemia samples were collected and processed between 2002 and 2014 according to Quebec Leukemia Cell Bank (BCLQ) standard operating procedures after obtaining written informed consent from all patients. Briefly, peripheral blood samples or bone marrow aspirates were collected in EDTA or heparin containing tubes, respectively, and white blood cells were subsequently obtained following Ficoll® isolation. For RT-qPCR experiments, NCI-H727 and HCT116 cell lines were used as positive controls. Cells designated for nucleic acid purification were frozen in TRIzol® reagent (Invitrogen®) and rapidly stored at −80° C. until RNA extraction. As part of the Leucegene project, the Research Ethics Boards of Université de Montréal and Maisonneuve-Rosemont Hospital approved all experiments.

RNA Isolation and Processing.

RNA was isolated from primary AML cells using TRIzol® reagent according to the manufacturer's instructions (Invitrogen®/Life Technologies®). For downstream sequencing applications an additional purification on RNeasy® mini columns (Qiagen®) was performed to obtain high quality RNA. Integrity verification of isolated RNA was performed on a Bioanalyzer® 2100 with a RIN>8 deemed acceptable. For RT-qPCR analyses, complementary DNA was generated from RNA using a Qiagen® QuantiTect® Reverse Transcription Kit (Qiagen®) according to manufacturer's protocols. For sequencing experiments, libraries were constructed with the TruSeq® RNA Sample Preparation Kit (Illumina®) according to manufacturer's protocols.

Quantitative PCR Analysis.

Quantitative PCR was performed using the TaqMan® Gene Expression system (Applied Biosystems®) on an Applied Biosystems® 7500 and in 0.2 ml 96-well polypropylene transparent PCR plates (Sarstedt®). Primer sequences were the following: ABL1-F 5′-TGGAGATAACACTCTAAGCATAACTAAAGGT-3′ (SEQ ID NO:4), ABL1-R 5′-GATGTAGTTGCTTGGGACCCA-3′ (SEQ ID NO:5), PAWR-F 5′-TGGTCAACATCCCTGCCG-3′ (SEQ ID NO:7), PAWR-R 5′-TTGCATCTTCTCGTTTCCGC-3′ (SEQ ID NO:8), HMGA2-F 5′-CACTTCAGCCCAGGGACAA-3′ (SEQ ID NO:1), HMGA2-R 5′-CTCACCGGTTGGTTCTTGCT-3′ (SEQ ID NO:2). FAM/ZEN/IBFQ probe sequences were the following: ABL1 5′-CCATTTTTGGTTTGGGCTTCACACCATT-3′ (SEQ ID NO:6), PAWR 5′-AGTACGAAGATGATGAAGCAGGGC-3′ (SEQ ID NO:9), HMGA2 5′-CTCAGAAGAGAGGACGCGGCC-3′ (SEQ ID NO:3). Plasmid standard curves were developed for ABL1 and PAWR on pMA-T vectors and HMGA2 on the pMA-RQ vector backbone. Data analysis was performed on the Applied Biosystems® 7500 software v2.0.5, with the threshold set at 0.1 and baseline set between cycles 3 and 15.

Sequencing and Bioinformatics Analysis.

Sequencing was performed on 437 AML specimens using an Illumina HiSeq® 2000 with 200 cycle-paired end runs. Sequence data were mapped to the reference genome hg19 using the Illumina Casava® 1.8.2 package and Elandv2 mapping software according to RefSeq annotations (UCSC, April 16th 2014). Transcript levels were given as Reads Per Kilobase per Million mapped reads (RPKM) and genes were annotated according to RefSeq annotations (UCSC, April 16th 2014).

Statistics.

Analysis of differential gene expression was performed with the Wilcoxon rank-sum test (Mann-Whitney) using the stats R package (http://cran.r-project.org/) with estimation of the False-discovery rate (FDR, q-value). In order to avoid issues with log-scale representation of RPKM values or normalized copy numbers equal to zero, a small constant (0.0001 or 0.01, respectively) was added to all expression values when log transformation was performed. For scatterplot visualizations, averages of groups were performed on log₁₀-transformed values to avoid overrepresentation of extremes. Survival analyses were performed in R using the CRAN package ‘survival’. A log-rank test was applied to compare the survival curves and determine if they were equivalent. The resulting p-value was reported on the corresponding Kaplan-Meier plots.

### Example 2: Genes Differentially Expressed in TP53-Mutant AML Compared to TP53-Wildtype AML

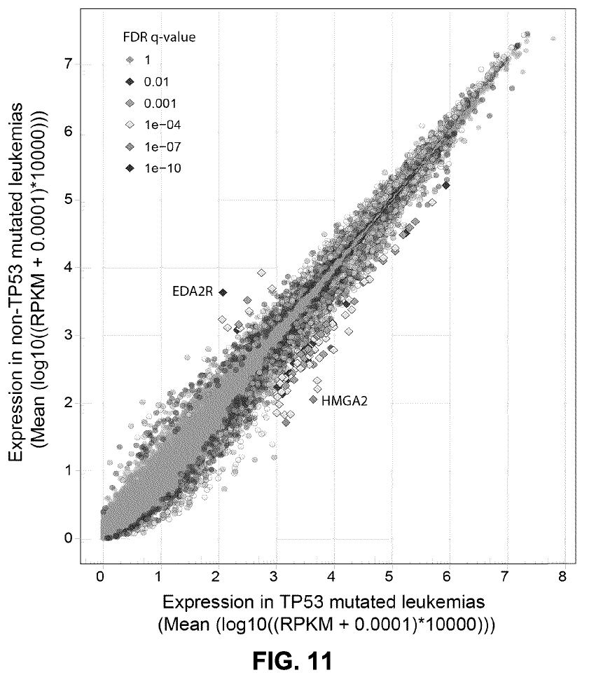

The gene expression signature of TP53-mutant AML, which is typically associated with a complex karyotype and poor outcome/prognosis, was determined. Many genes were differentially expressed in TP53-mutant AML compared to TP53-wildtype AML. The genes with the greatest differential overexpression (HMGA2) and underexpression (EDA2R) in TP53-mutant AML are highlighted in FIG. 11, and genes showing significant overexpression and underexpression in TP53-mutant AML relative to TP53-wildtype AML (volcano difference 0.7 or ≤−0.7, respectively) are depicted in Table 2A and 2B, respectively.

TABLE 2A
Genes showing significant overexpression in
TP53-mutant AML relative to TP53-wildtype AML

| | Ensembl | | | | |
|---|---|---|---|---|---|
| Overexpressed | Gene ID | FDR | TP53 mutated | TP53 wt | volcano |
| gene | (ENSG) | q-value | (mean(log10)) | (mean(log10)) | difference |
| HMGA2 | 00000149948 | 3.71E−08 | 3.64 | 2.05 | 1.59 |
| MIR451A | 00000273915 | 3.56E−06 | 3.71 | 2.21 | 1.50 |
| LOC644554 | 00000267640 | 3.15E−08 | 3.16 | 1.71 | 1.45 |
| MIR144 | 00000277441 | 6.45E−06 | 3.24 | 1.83 | 1.41 |
| CYYR1 | 00000166265 | 2.42E−07 | 3.70 | 2.33 | 1.37 |
| FGF16 | 00000196468 | 1.51E−06 | 3.14 | 1.82 | 1.31 |
| MIR503HG | 00000223749 | 8.39E−07 | 3.99 | 2.78 | 1.21 |
| DEFA8P | 00000223629 | 2.41E−05 | 4.25 | 3.06 | 1.19 |
| LOC441666 | 00000215146 | 1.68E−07 | 3.00 | 1.85 | 1.15 |
| ZNF229 | 00000278318 | 7.74E−11 | 3.94 | 2.81 | 1.13 |
| DDIT4L | 00000145358 | 0.000157363 | 3.81 | 2.72 | 1.09 |
| MYCT1 | 00000120279 | 1.51E−05 | 3.74 | 2.66 | 1.08 |
| DLK1 | 00000185559 | 0.000236516 | 3.64 | 2.57 | 1.07 |
| ZNF835 | 00000127903 | 1.72E−07 | 3.04 | 1.98 | 1.07 |
| ART4 | 00000111339 | 7.33E−05 | 3.57 | 2.57 | 1.00 |
| KIF5A | 00000155980 | 1.35E−06 | 3.18 | 2.18 | 1.00 |
| NMU | 00000109255 | 0.001250977 | 3.10 | 2.13 | 0.97 |
| LINC00570 | 00000224177 | 0.00010146 | 3.30 | 2.34 | 0.96 |
| NKAIN2 | 00000188580 | 1.77E−05 | 3.02 | 2.09 | 0.93 |
| TACSTD2 | 00000184292 | 4.63E−05 | 4.30 | 3.38 | 0.93 |
| TPD52L1 | 00000111907 | 3.39E−06 | 3.08 | 2.16 | 0.92 |
| MEG3 | 00000214548 | 6.54E−05 | 3.45 | 2.52 | 0.92 |
| CHRDL1 | 00000101938 | 0.00192006 | 3.54 | 2.63 | 0.91 |
| VWDE | 00000146530 | 2.60E−05 | 3.42 | 2.51 | 0.91 |
| PKDCC | 00000162878 | 0.000567351 | 3.16 | 2.25 | 0.90 |
| KLHDC8A | 00000162873 | 0.000136337 | 3.46 | 2.56 | 0.90 |
| CDC42BPA | 00000143776 | 1.23E−05 | 3.85 | 2.95 | 0.90 |
| PAWR | 00000177425 | 1.51E−05 | 4.19 | 3.30 | 0.89 |
| HBZ | 00000130656 | 0.016100874 | 3.66 | 2.77 | 0.89 |
| TRPM6 | 00000119121 | 2.40E−07 | 3.25 | 2.37 | 0.89 |
| GIPC2 | 00000137960 | 2.51E−07 | 3.01 | 2.13 | 0.88 |
| PLSCR4 | 00000114698 | 8.28E−05 | 3.57 | 2.69 | 0.88 |
| SEMA3C | 00000075223 | 3.44E−06 | 3.66 | 2.78 | 0.87 |
| COL17A1 | 00000065618 | 1.08E−05 | 3.99 | 3.12 | 0.87 |
| HIST3H2BB | 00000196890 | 7.18E−05 | 3.13 | 2.28 | 0.85 |
| ZNF727P | 00000214652 | 0.001354829 | 3.20 | 2.35 | 0.85 |
| CRISP3 | 00000096006 | 0.000689969 | 4.34 | 3.50 | 0.85 |
| PAQR9 | 00000188582 | 0.000739117 | 3.59 | 2.74 | 0.84 |
| CCDC8 | 00000169515 | 0.00021802 | 3.27 | 2.43 | 0.84 |
| HIST1H3E | 00000274750 | 2.81E−06 | 3.73 | 2.90 | 0.83 |
| PKLR | 00000143627 | 0.000273639 | 3.85 | 3.03 | 0.83 |
| ZNF883 | 00000228623 | 1.57E−05 | 3.34 | 2.51 | 0.83 |
| ZNF90 | 00000213988 | 1.98E−09 | 3.98 | 3.15 | 0.83 |
| ESPN | 00000187017 | 9.12E−05 | 3.85 | 3.03 | 0.83 |
| HIST1H3G | 00000273983 | 0.00039197 | 3.48 | 2.66 | 0.82 |
| FGD5 | 00000154783 | 0.000103553 | 3.59 | 2.77 | 0.82 |
| ANKRD65 | 00000235098 | 5.60E−05 | 3.37 | 2.56 | 0.82 |
| CEACAM6 | 00000086548 | 0.000271307 | 5.05 | 4.24 | 0.81 |
| NFIA | 00000162599 | 9.37E−06 | 3.82 | 3.01 | 0.81 |
| LILRB5 | 00000273991 | 0.001815013 | 3.25 | 2.44 | 0.80 |
| FAM83F | 00000133477 | 9.74E−05 | 3.36 | 2.55 | 0.80 |
| LOC101927720 | 00000266916 | 4.83E−07 | 3.97 | 3.17 | 0.80 |
| BEND4 | 00000188848 | 0.000133886 | 3.87 | 3.07 | 0.80 |
| DPPA4 | 00000121570 | 0.003541264 | 3.97 | 3.17 | 0.79 |
| CLEC4D | 00000166527 | 1.29E−05 | 4.61 | 3.82 | 0.79 |
| OSBPL6 | 00000079156 | 6.68E−05 | 3.15 | 2.37 | 0.79 |
| SLC24A5 | 00000188467 | 0.001949244 | 3.02 | 2.23 | 0.79 |
| FERMT1 | 00000101311 | 0.001478813 | 3.21 | 2.43 | 0.78 |
| MIR6730 | 00000276830 | 0.00448761 | 3.12 | 2.35 | 0.77 |
| MYT1 | 00000196132 | 3.07E−06 | 3.06 | 2.29 | 0.77 |
| CHI3L1 | 00000133048 | 6.10E−05 | 5.05 | 4.29 | 0.77 |
| MFAP2 | 00000117122 | 3.32E−05 | 3.36 | 2.59 | 0.77 |
| APOC1 | 00000130208 | 0.000337606 | 4.58 | 3.82 | 0.76 |
| MYZAP | 00000263155 | 0.000853269 | 3.55 | 2.79 | 0.76 |
| OLFM4 | 00000102837 | 0.001768516 | 4.21 | 3.46 | 0.75 |
| CA4 | 00000167434 | 0.001275227 | 3.64 | 2.89 | 0.75 |
| CLEC9A | 00000197992 | 0.000112148 | 3.76 | 3.01 | 0.75 |
| ZNF826P | 00000231205 | 1.12E−05 | 3.38 | 2.63 | 0.75 |
| CEACAM8 | 00000124469 | 0.000476371 | 4.84 | 4.10 | 0.75 |
| KCNJ15 | 00000157551 | 0.000106494 | 3.51 | 2.77 | 0.74 |
| MMP8 | 00000118113 | 0.001596518 | 5.24 | 4.51 | 0.73 |
| APP | 00000142192 | 1.99E−06 | 5.69 | 4.96 | 0.73 |
| HBE1 | 00000213931 | 0.01954452 | 3.44 | 2.71 | 0.73 |
| HIST3H2A | 00000181218 | 3.87E−06 | 4.52 | 3.79 | 0.73 |
| HBG1 | 00000213934 | 0.000992641 | 5.40 | 4.68 | 0.73 |
| RAB3IL1 | 00000167994 | 2.60E−05 | 4.25 | 3.53 | 0.73 |
| FAM178B | 00000168754 | 0.001536478 | 3.55 | 2.82 | 0.73 |
| ITLN1 | 00000179914 | 3.38E−06 | 3.73 | 3.01 | 0.72 |
| BTNL10 | 00000215811 | 0.00146285 | 3.30 | 2.58 | 0.72 |
| FZD6 | 00000164930 | 4.87E−08 | 4.50 | 3.78 | 0.72 |
| ZNF626 | 00000188171 | 9.35E−08 | 3.70 | 2.98 | 0.72 |
| HBG2 | 00000196565 | 0.001248089 | 5.93 | 5.21 | 0.72 |
| LCN2 | 00000148346 | 0.000377753 | 5.31 | 4.59 | 0.72 |
| ZNF462 | 00000148143 | 9.23E−05 | 3.09 | 2.38 | 0.72 |
| CLCN4 | 00000073464 | 9.01E−06 | 3.69 | 2.97 | 0.71 |
| PGLYRP1 | 00000008438 | 0.000347922 | 5.19 | 4.48 | 0.71 |
| GAL | 00000069482 | 8.40E−05 | 3.65 | 2.95 | 0.71 |
| IFI27 | 00000165949 | 2.51E−05 | 4.85 | 4.14 | 0.71 |
| TUSC1 | 00000198680 | 0.000612212 | 3.86 | 3.16 | 0.70 |
| LINC01133 | 00000224259 | 0.002219556 | 3.49 | 2.78 | 0.70 |
| GAD1 | 00000128683 | 0.000325874 | 3.42 | 2.72 | 0.70 |
| SLC16A10 | 00000112394 | 4.81E−08 | 3.43 | 2.73 | 0.70 |

TABLE 2B
Genes showing the most significant underexpression
in TP53-mutant AML relative to TP53-wildtype AML

| | Ensembl | | | | |
|---|---|---|---|---|---|
| Underexpressed | Gene ID | FDR | TP53 mutated | TP53 wt | volcano |
| gene | (ENSG) | q-value | (mean(log10)) | (mean(log10)) | difference |
| EDA2R | 00000131080 | 3.01E−14 | 2.07 | 3.63 | −1.56 |
| PCAT18 | 00000265369 | 1.24E−07 | 2.74 | 3.92 | −1.18 |
| DAPL1 | 00000163331 | 7.76E−05 | 2.06 | 3.23 | −1.17 |
| IRX3 | 00000177508 | 0.000241063 | 2.50 | 3.52 | −1.02 |
| TBC1D29 | 00000266733 | 1.72E−07 | 2.15 | 3.11 | −0.96 |
| IRX5 | 00000176842 | 3.67E−05 | 2.52 | 3.35 | −0.83 |
| CLEC14A | 00000176435 | 0.000937071 | 2.35 | 3.16 | −0.81 |
| HOXB.AS3 | 00000233101 | 0.012935295 | 2.54 | 3.32 | −0.77 |
| LOC339862 | 00000274840 | 1.33E−05 | 2.92 | 3.69 | −0.77 |
| C20orf166.AS1 | 00000174403 | 0.003625215 | 2.32 | 3.08 | −0.76 |
| LOC101927438 | 00000234572 | 1.39E−06 | 2.37 | 3.10 | −0.73 |
| HOXB6 | 00000108511 | 0.0110241 | 2.92 | 3.64 | −0.72 |

FDR q value = False Discovery Rate; TP53 mutated (mean(log10)) = Mean RPKM expression in TP53 mutated AML (log10 transformed); TP53 wt (mean(log10)) = Mean RPKM expression in TP53 wild-type AML (log10 transformed); Volcano difference = log10 transformed difference between TP53 mutated vs. wild-type

### Example 3: HMGA2 is a Prognostic Marker for AML, Including Intermediate-Risk AML

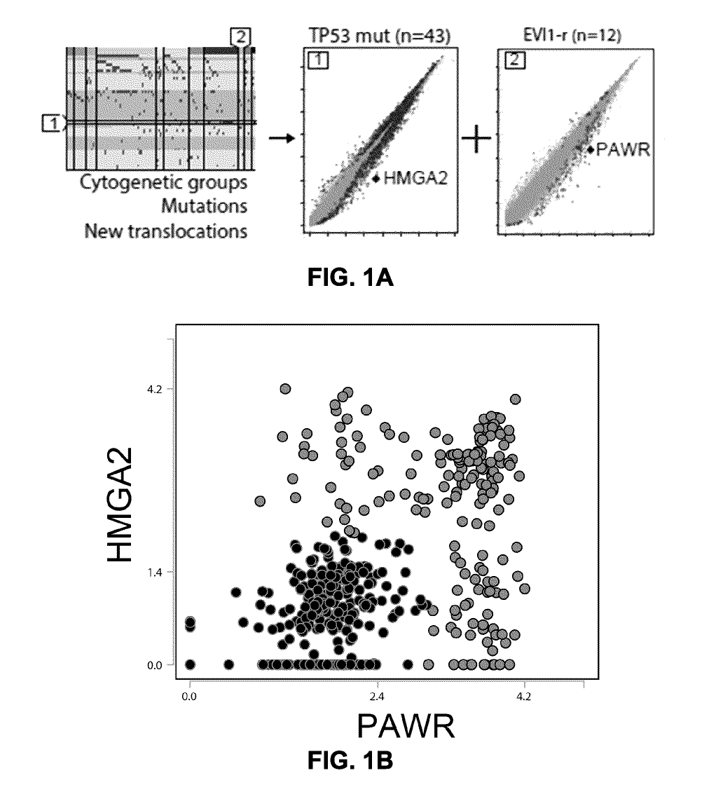

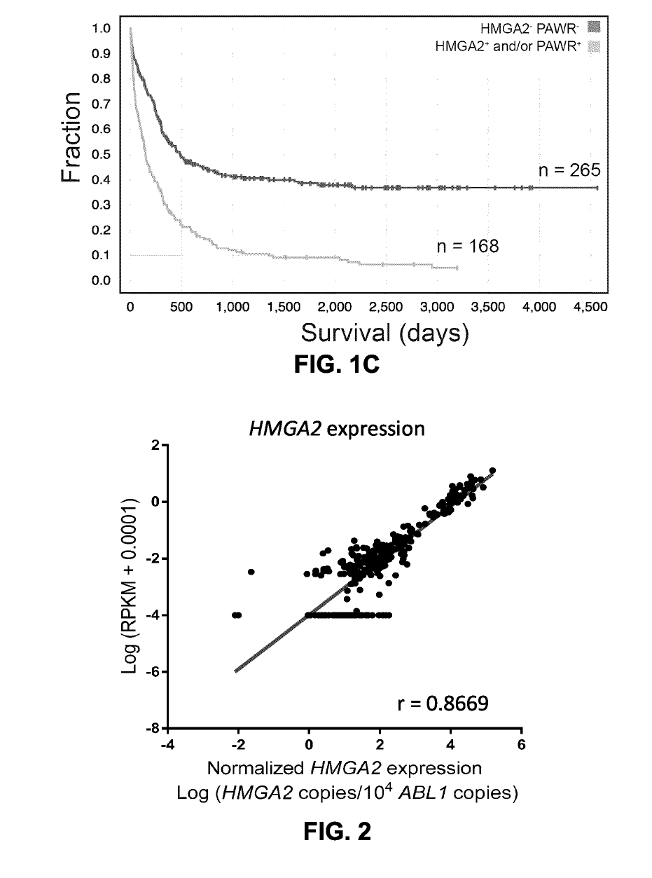

FIGS. 1A to 1C show the approach used to identify prognostic markers for AML. A total of 437 primary human AML specimens were subjected to a refined process to identify novel indications of prognostic/diagnostic value. Mutations, classical cytogenetic groups, novel fusions and the transcriptomic signatures were first identified to separate AML specimens into appropriate subtypes. As shown in Example 2 above, a comparison between the transcriptomes of TP53 mut AML (associated with poor prognosis) and the rest of the cohort identified HMGA2 as amongst the most differentially expressed gene in this subtype. PAWR was previously identified as one of the most differentially expressed gene in the EVI1-r AML genetic subtype (Lavallée V P, et al. Blood. 2015 Jan. 1; 125(1):140-3). The expression levels of these genes were subsequently explored in the complete cohort to assess their prognostic value in AML. The resulting scatterplot comparing HMGA2 and PAWR expression in the entire cohort is seen in FIG. 1B. This analysis resultantly identified additional samples expressing one or both markers but not associated to TP53 mut or EVI1-r genetic aberrations. These new groups (HMGA2+ and/or PAWR+ vs. HMGA2-PAWR−) were further examined via Kaplan-Meier survival curve analysis. As observed in FIG. 1C, the survival curve of this new group was found to associate to adverse outcome. Therefore, this approach is thereby able to identify a significant number of patients with poor prognosis (168 HMGA2+ and/or PAWR+ compared to 55 TP53 mutated or EVI1-rearranged samples).

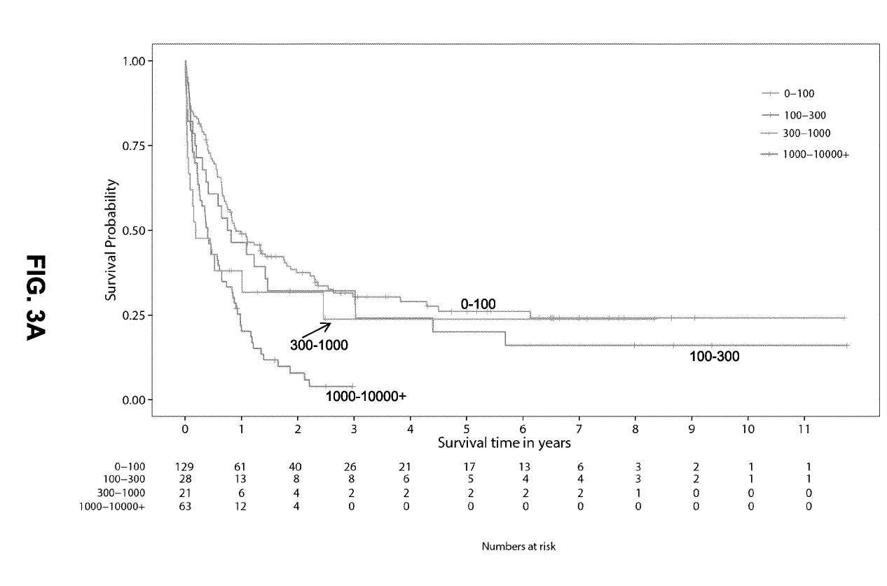

It was next assessed whether the transcriptome data could be reproduced in a RT-qPCR assay. FIG. 2 shows the good correlation (r=0.8669) in HMGA2 expression levels detected using either RNA-Seq transcriptome data (log RPKM) or the RT-qPCR assay described herein (normalized copy number, NCN). The prognostic value of HMGA2 in AML patients was thus evaluated using a RT-qPCR assay.

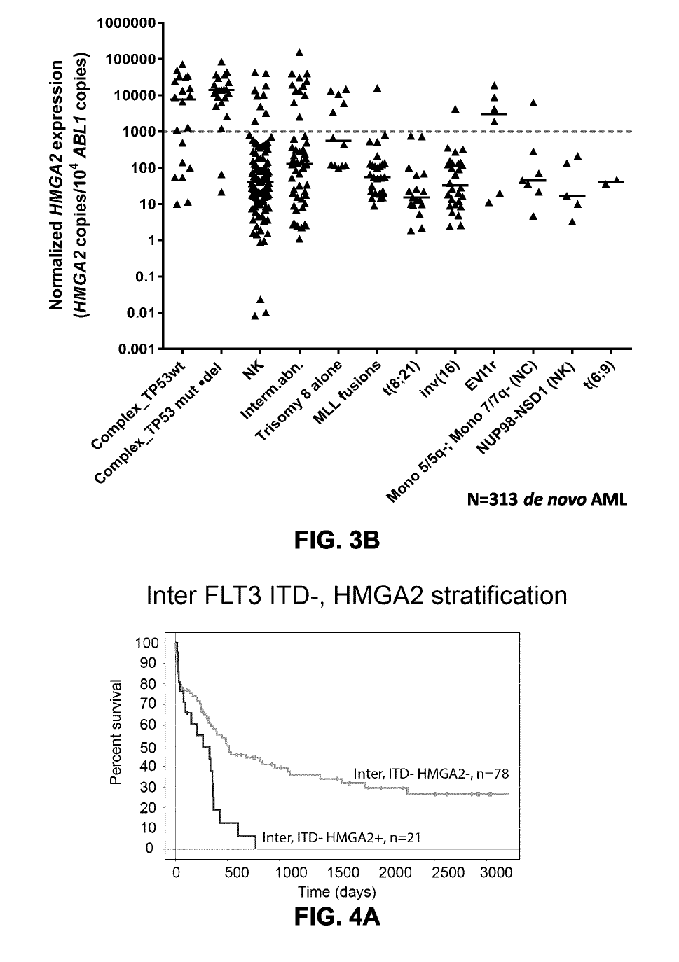

A significant difference in overall survival was observed in samples expressing greater than or equal to 1000 HMGA2 normalized copy numbers, thereby identifying a potential cut-off value (FIG. 3A). FIG. 3B shows the expression levels of genetic subtypes within the Leucegene de novo AML cohort as determined using the HMGA2 RT-qPCR assay, which demonstrate the large dynamic range in HMGA2 expression levels between genetic subtypes in the Leucegene de novo AML cohort, and that greater than 1000 normalized copy number expression of HMGA2 was typically associated with genetic subtypes with known adverse clinical outcome. However, several additional specimens that were either normal karyotype, intermediate abnormal AML, or otherwise not associated with known genetic subgroups of poor clinical outcome (typically referred to as “intermediate-risk” AMLs) were shown to have HMGA2 expression values above the set cut-off value, suggesting that HMGA2 expression could potentially be used to predict poor clinical outcome in AML patients, including intermediate-risk AML patients.

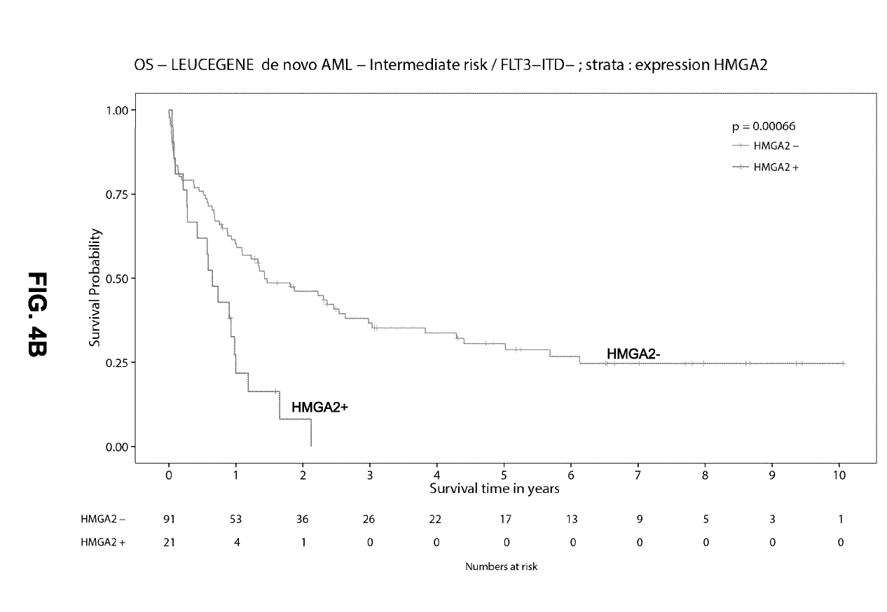

The presence of FLT3 aberrations, notably internal tandem duplication (ITD), is typically associated with an unfavorable clinical outcome. However, the prediction of the clinical outcome (i.e. good vs. poor prognosis/outcome) of FLT3-ITD negative AML patients is much more difficult. Thus, it was next assessed whether HMGA2 expression could be used to re-stratify these “intermediate-risk” patients into good and poor outcome. FIGS. 4A and 4B show the overall survival curves according to HMGA2 expression in Leucegene de novo AML Intermediate Risk FLT3-ITD-negative cohort determined by RNA-Seq (FIG. 4A) and RT-qPCR (FIG. 4B), which demonstrate poor overall survival in specimens whose HMGA2 expression is >0.1 RPKM (FIG. 4A) or 1000 normalized HMGA2 copy number per 10⁴ ABL1 copy number (FIG. 4B). These results confirm that HMGA2 expression may be used as a marker to predict the clinical outcome in intermediate-risk FLT3-ITD-negative patients.

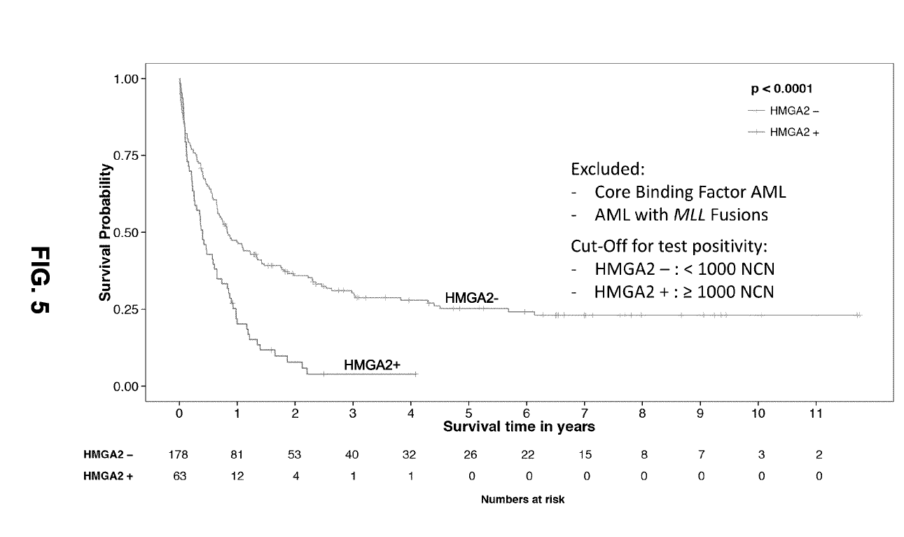

It was next assessed whether HMGA2 expression may be used as a marker to predict the clinical outcome in the general AML patient population, i.e. irrespective of the AML subgroup (only core binding factor AMLs (inv(16), t(8;21)), which have favorable risk status and AML with MLL fusions, for which the HMGA2 test is less informative, were excluded from the analysis). FIG. 5 depicts the overall survival curves according to HMGA2 expression determined by RT-qPCR in the Leucegene AML cohort, which demonstrates the poor overall survival by specimens whose HMGA2 expression is 1000 normalized copy number (NCN) relative to specimens<1000 NCN. These results confirm that HMGA2 expression may be used as a marker to predict the clinical outcome in AML patients.

### Example 4: HMGA2 Expression in Combination with PAWR Expression as Prognostic Markers for AML, Including Intermediate-Risk AML

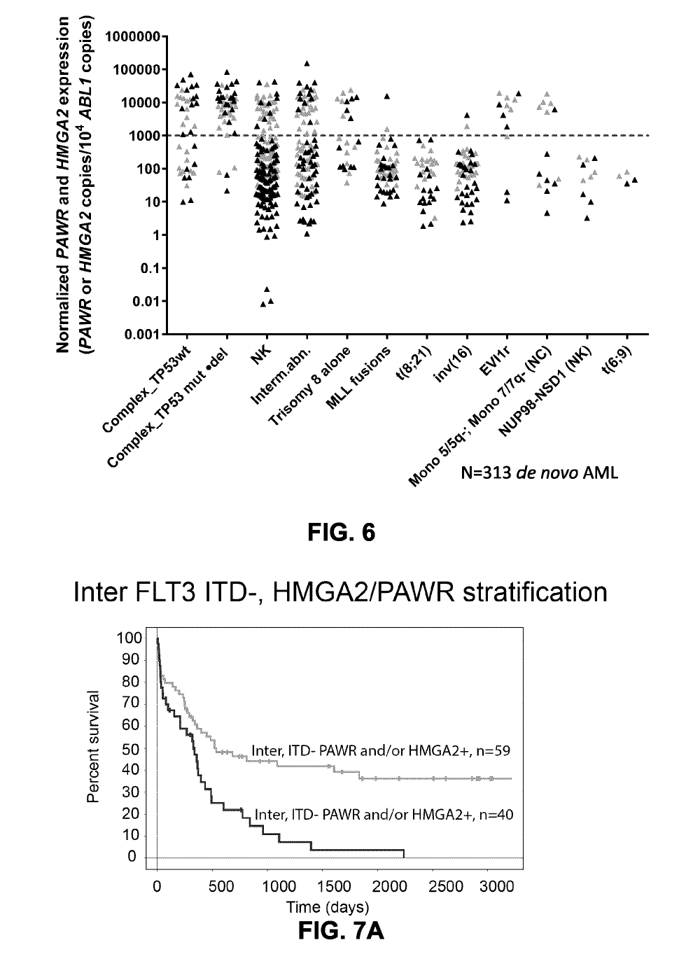

It was tested whether the combination of HMGA2 expression with an additional marker, PAWR (which is highly expressed in EVI1-r AMLs, associated with poor outcome), would provide an improved prognostic power relative to HMGA2 expression only. FIG. 6 shows the combined HMGA2 and PAWR and analysis using RT-qPCR in the Leucegene AML cohort, which allowed the identification of a cohort with robust expression of at least one of either marker.

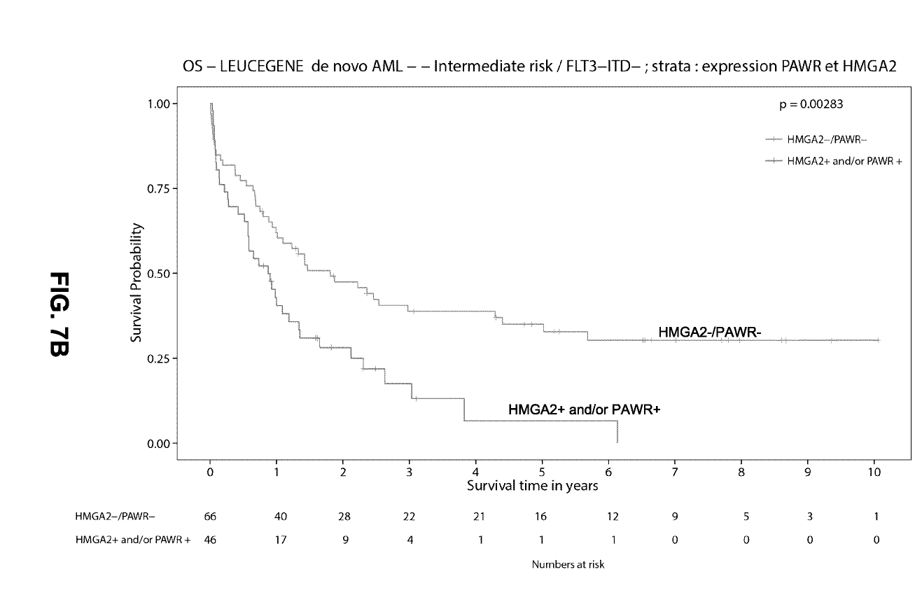

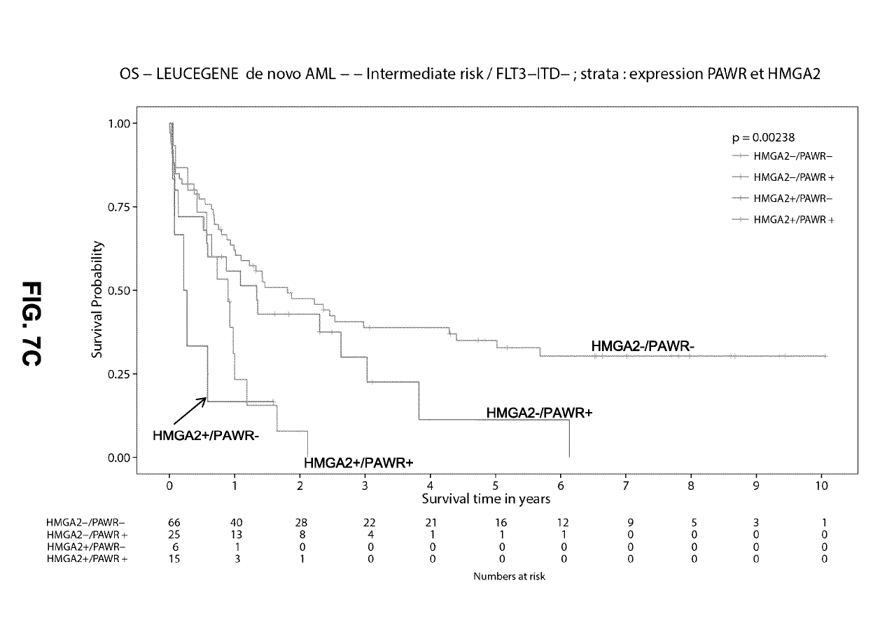

The overall survival according to HMGA2/PAWR expression was determined in the AML Intermediate Risk FLT3-ITD-negative cohort. Kaplan-Meier survival probability analysis on Intermediate Risk FLT3-ITD-negative (ITD−) AML based upon HMGA2 and/or PAWR expression determined by RNA-Seq (FIG. 7A) and RT-qPCR (FIGS. 7B and 7C) shows poor overall survival by specimens whose HMGA2 and/or PAWR expression is >0.1 RPKM or 1 RPKM, respectively (FIG. 7A) or 1000 normalized values of HMGA2/PAWR copy number per 10⁴ ABL1 copy number (FIGS. 7B and 7C). Therefore, the combination of HMGA2 and PAWR expression can act to re-stratify approximately 40% of patients in the Intermediate Risk, FLT3-ITD-negative cohort with poor overall survival. Specimens deemed to be singly positive in expression 1000 NCN) for either HMGA2 or PAWR are deemed to have worse overall survival compared to specimens<1000 NCN for either marker. Therefore, use of these markers can act to identify patients with poor overall survival and re-stratify the Intermediate Risk FLT3-ITD-negative cohort.

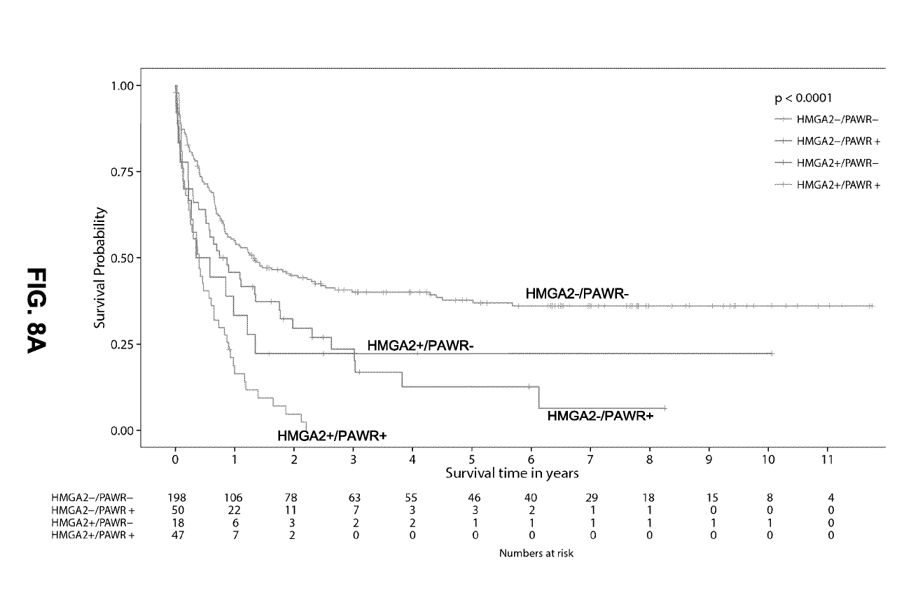

It was next assessed whether HMGA2 and PAWR expression may be used as markers to predict the clinical outcome in the general AML patient population, i.e. irrespective of the AML subgroup. Only core binding factor AMLs (inv(16), t(8;21)), which have favorable risk status, and AML with MLL fusions, for which the HMGA2 test is less informative, were excluded from the analysis). FIG. 8A depicts the overall survival curves according to HMGA2 and PAWR expression determined by RT-qPCR in the Leucegene AML cohort, which demonstrates the poorest overall survival by specimens double positive for HMGA2 and PAWR expression, i.e. 1000 normalized values of HMGA/PAWR copy number per 10⁴ ABL1 copy number (NCN). Specimens deemed to be singly positive in expression 1000 NCN) for either HMGA2 or PAWR are deemed to have worse overall survival compared to specimens<1000 NCN for either marker. Similar results were obtained when AML patients with a favourable prognosis (t(8;21) and inv(16)) and for which the HMGA2-PAWR test is less informative (AML with MLL fusions) were excluded from the survival analysis (FIG. 8B).These results confirm that the combination of HMGA2 and PAWR expression levels may be used as markers to predict the clinical outcome in AML patients, and has a stronger prognostic power/value than either marker used individually.

### Example 5: Further Validation of the HMGA2 and PAWR RT-qPCR Tests

The HMGA2 and PAWR RT-qPCR tests described above were validated in a second laboratory (laboratory of Professor David Grimwade at the Department of Medical and Molecular Genetics, King's College London), using 268 AML samples from the UK National Cancer Research Institute (NCRI) AML17 trial cohort (treated patients younger than 60 years of age) (aml17.cardiff.ac.uk, Ivey et al., N Engl J Med 2016; 374: 422-433). The intra-laboratory performance specifications are listed in Table 3.

TABLE 3
Analytical validation performance specifications
of HMGA2 and PAWR prognostic tests.

| Parameter | HMGA2 test | PAWR test |
|---|---|---|
| Specificity (%) | 100 | 100 |
| PCR Efficiency (%) | 100.55 ± 1.63 | 99.49 ± 0.93 |
| (mean ± SD) | | |
| Linearity R² (mean ± SD) | 0.999 ± 0.002 | 0.999 ± 0.001 |
| Analytical sensitivity | LoQ: 100 | LoQ: 100 |
| (LoQ; copies/5 μl) | | |
| Precision - Repeatability | 6.71 (2.59-16.0) | 4.55 (1.76-10.86) |
| (within-run) | | |
| copies/5 μl, % CV (95% CI) | | |
| Intermediate Precision | 8.61 (4.84-12.38) | 4.86 (2.73-6.99) |
| (day-to-day) | | |
| copies/5 μl, % CV (95% CI) | | |
| Ruggedness - MasterMix lot-to- | 8.71 (6.01-11.41) | 4.56 (3.15-5.97) |
| lot, copies/5 μl, %CV (95% CI) | | |
| Ruggedness - system to system, | 8.65 (5.97-11.32) | 5.38 (3.73-7.09) |
| copies/5 μl, % CV (95% CI) | | |
| Reportable Range (copies/5 μl) | 10² to 10⁶ | 10² to 10⁶ |

SD, Standard Deviation; LoQ, Limit of Quantification; CV, Coefficient of Variation; CI, Confidence Interval

The HMGA2 and PAWR RT-qPCR assay cut-offs for clinical outcome prediction were refined in the final AML cohort (hereafter called the test cohort) which comprises 358 AML patients treated with curative intent (263 patients of the Leucegene cohort and 95 patients with de novo AML from an independent BCLQ cohort). Using ROC curves (Kroll, M H. et al. Assessment of the diagnostic accuracy of laboratory tests using receiver operating characteristic curves; approved guideline—second edition. NCCLS; 2011. CLSI document EP24-A2) and Youden's index (Youden, W. J. (1950), Cancer 3: 32-35; Schisterman et al., (2005), Epidemiology 16: 73-81), it was determined that the optimal HMGA2 and PAWR test cut-offs are 1100 and 950 normalized copy number (NCN), respectively (Table 4).

TABLE 4
Performance results of HMGA2 and PAWR tests and cut-
off values for outcome prediction in AML patients.

| | | | | | | Youden's | |
|---|---|---|---|---|---|---|---|
| | End-point | | | | | index | Cut-Off |
| Test | (yrs) | Sensitivity | Specificity | PPV | NPV | (NCN) | (NCN) |
| HMGA2 | 3 | 30.8% | 92.5% | 90.4% | 37.0% | 1099 | 1100 |
| | 5 | 30.5% | 93.8% | 94.4% | 27.9% | 1099 | |
| PAWR | 3 | 41.1% | 85.1% | 86.3% | 38.8% | 937 | 950 |
| | 5 | 41.7% | 89.1% | 93.0% | 30.5% | 937 | |

PPV: Positive Predictive Value;
NPV: Negative Predictive Value;
NCN, Normalized Copy Number

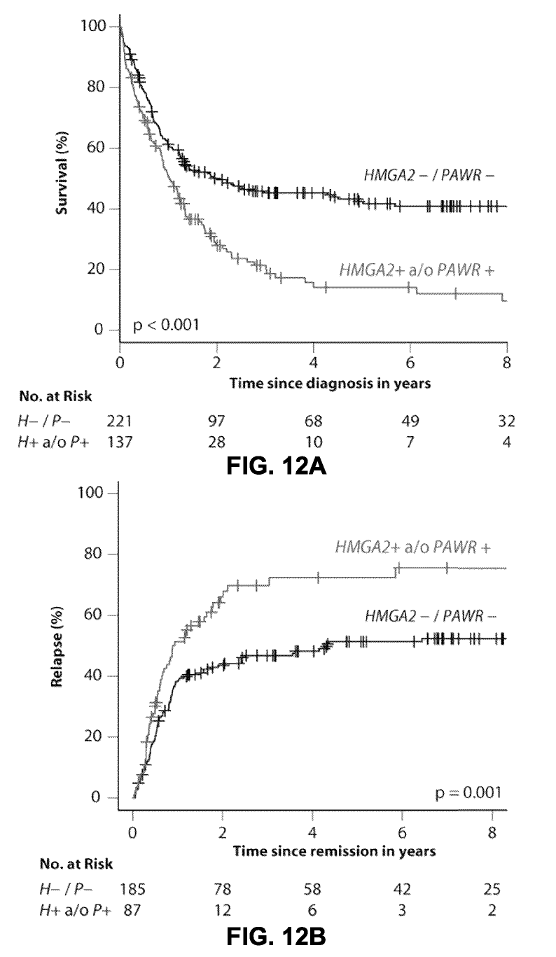

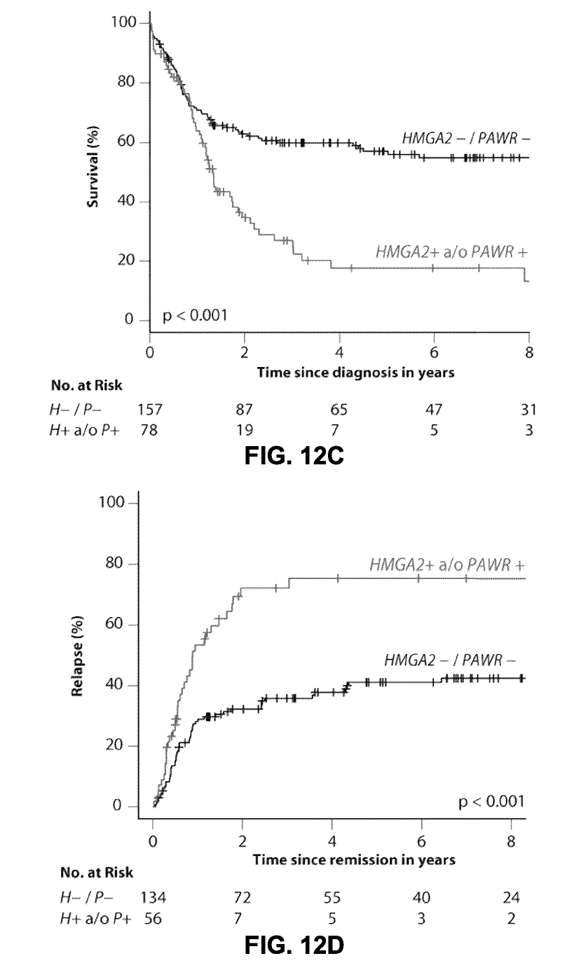

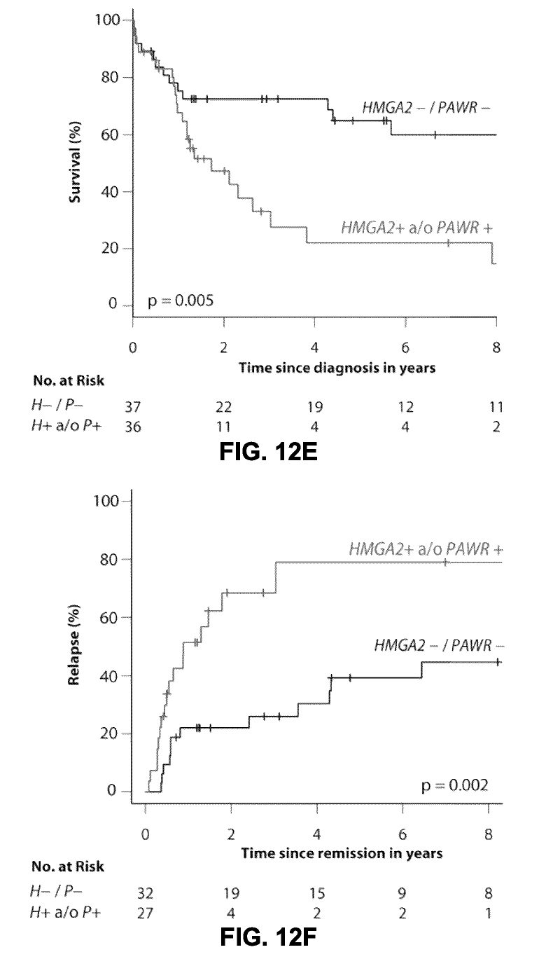

Further analyses revealed that HMGA2 and PAWR gene expression as measured using the HMGA2/PAWR RT-qPCR assay is highly predictive of overall survival and cumulative incidence of relapse in AML patients (FIGS. 12A and 12B). This predictive value was shown to be greater when only younger AML patients (i.e. less than 60 years old, and more particularly from 17 to 59 years old) were studied (FIGS. 12C and 12D). Moreover, the HMGA2-PAWR gene expression signature was shown to improve risk stratification in the intermediate-risk genetic AML patients younger than 60 years of age (FIGS. 12E and 12F).

Although the present invention has been described hereinabove by way of specific embodiments thereof, it can be modified, without departing from the spirit and nature of the subject invention as defined in the appended claims. The scope of the claims should not be limited by the preferred embodiments set forth in the examples, but should be given the broadest interpretation consistent with the description as a whole.

What is claimed is:

1. A method for the disease prognosis of a patient suffering from acute myeloid leukemia (AML), said method comprising: measuring the level of expression of High Mobility Group AT-hook 2 (HMGA2) in a biological sample comprising leukemic cells from said patient; and comparing said level of expression to a threshold reference level, wherein a level of expression that is higher than threshold reference level is indicative of a poor disease prognosis.

2. The method of claim 1, wherein said level of expression is measured at the nucleic acid level.

3. The method of claim 2, wherein said method comprises amplifying a nucleic acid encoding HMGA2 using a first HMGA2 primer and a second HMGA2 primer.

4. The method of claim 3, wherein said first HMGA2 primer comprises at least 10 nucleotides of the sequence 5′-CACTTCAGCCCAGGGACAA-3′ (SEQ ID NO: 1).

5. The method of claim 4, wherein said first HMGA2 primer comprises at least 10 contiguous nucleotides of the sequence 5′-CACTTCAGCCCAGGGACAA-3′ (SEQ ID NO: 1).

6. The method of claim 5, wherein said first HMGA2 primer comprises the sequence 5′-CACTTCAGCCCAGGGACAA-3′ (SEQ ID NO: 1).

7. The method of any one of claims 3 to 6, wherein said second HMGA2 primer comprises at least 10 nucleotides of the sequence 5′-CTCACCGGTTGGTTCTTGCT-3′ (SEQ ID NO: 2).

8. The method of claim 7, wherein said second HMGA2 primer comprises at least 10 contiguous nucleotides of the sequence 5′-CTCACCGGTTGGTTCTTGCT-3′ (SEQ ID NO: 2).

9. The method of claim 8, wherein said second HMGA2 primer comprises the sequence 5′-CTCACCGGTTGGTTCTTGCT-3′ (SEQ ID NO: 2).

10. The method of any one of claims 2 to 9, wherein said method comprises detecting the nucleic acid encoding HMGA2 using a HMGA2 probe.

11. The method of claim 10, wherein said HMGA2 probe comprises at least about 10 nucleotides of the sequence 5′-CTCAGAAGAGAGGACGCGGCC-3′ (SEQ ID NO: 3).

12. The method of claim 11, wherein said HMGA2 probe comprises at least about 10 contiguous nucleotides of the sequence 5′-CTCAGAAGAGAGGACGCGGCC-3′ (SEQ ID NO: 3).

13. The method of claim 12, wherein said HMGA2 probe comprises the sequence 5′-CTCAGAAGAGAGGACGCGGCC-3′ (SEQ ID NO: 3).

14. The method of any one of claims 2 to 13, wherein the level of expression of HMGA2 is measured by reverse transcription polymerase chain reaction (RT-PCR).

15. The method of any one of claims 1 to 14, wherein said method further comprises normalizing the level of expression of HMGA2 based on the level of expression of a housekeeping gene.

16. The method of claim 15, wherein said housekeeping gene is ABL1.

17. The method of claim 16, wherein said method comprises amplifying a nucleic acid encoding ABL1 using a first ABL1 primer and a second ABL1 primer.

18. The method of claim 17, wherein said first ABL1 primer comprises at least 10 nucleotides of the sequence 5′-TGGAGATAACACTCTAAGCATAACTAAAGGT-3′ (SEQ ID NO: 4).

19. The method of claim 18, wherein said first ABL1 primer comprises at least 10 contiguous nucleotides of the sequence 5′-TGGAGATAACACTCTAAGCATAACTAAAGGT-3′ (SEQ ID NO: 4).

20. The method of claim 19, wherein said first ABL1 primer comprises the sequence 5′-TGGAGATAACACTCTAAGCATAACTAAAGGT-3′ (SEQ ID NO: 4).

21. The method of any one of claims 17 to 20, wherein said second ABL1 primer comprises at least 10 nucleotides of the sequence 5′-GATGTAGTTGCTTGGGACCCA-3′ (SEQ ID NO: 5).

22. The method of claim 21, wherein said second ABL1 primer comprises at least 10 contiguous nucleotides of the sequence 5′-GATGTAGTTGCTTGGGACCCA-3′ (SEQ ID NO: 5).

23. The method of claim 22, wherein said second ABL1 primer comprises the sequence 5′-GATGTAGTTGCTTGGGACCCA-3′ (SEQ ID NO: 5).

24. The method of any one of claims 16 to 23, wherein said method comprises detecting the nucleic acid encoding ABL1 using an ABL1 probe.

25. The method of claim 24, wherein said ABL1 probe comprises at least 10 nucleotides of the sequence 5′-CCATTTTTGGTTTGGGCTTCACACCATT-3′ (SEQ ID NO: 6).

26. The method of claim 25, wherein said ABL1 probe comprises at least 10 contiguous nucleotides of the sequence 5′-CCATTTTTGGTTTGGGCTTCACACCATT-3′ (SEQ ID NO: 6).

27. The method of claim 26, wherein said ABL1 probe comprises the sequence 5′-CCATTTTTGGTTTGGGCTTCACACCATT-3′ (SEQ ID NO: 6).

28. The method of any one of claims 1 to 27, further comprising measuring the level of expression of at least one additional prognostic marker gene in said biological sample.

29. The method of claim 28, wherein said at least one additional prognostic marker gene is PRKC Apoptosis WT1 Regulator (PAWR).

30. The method of claim 29, wherein said method comprises amplifying a nucleic acid encoding PAWR using a first PAWR primer and a second PAWR primer.

31. The method of claim 30, wherein said first PAWR primer comprises at least 10 nucleotides of the sequence 5′-TGGTCAACATCCCTGCCG-3′ (SEQ ID NO: 7).

32. The method of claim 31, wherein said first PAWR primer comprises at least 10 contiguous nucleotides of the sequence 5′-TGGTCAACATCCCTGCCG-3′ (SEQ ID NO: 7).

33. The method of claim 32, wherein said first PAWR primer comprises the sequence 5′-TGGTCAACATCCCTGCCG-3′ (SEQ ID NO: 7).

34. The method of any one of claims 30 to 33, wherein said second PAWR primer comprises at least 10 nucleotides of the sequence 5′-TTGCATCTTCTCGTTTCCGC-3′ (SEQ ID NO: 8).

35. The method of claim 34, wherein said second PAWR primer comprises at least 10 contiguous nucleotides of the sequence 5′-TTGCATCTTCTCGTTTCCGC-3′ (SEQ ID NO: 8).

36. The method of claim 35, wherein said second PAWR primer comprises the sequence 5′-TTGCATCTTCTCGTTTCCGC-3′ (SEQ ID NO: 8).

37. The method of any one of claims 30 to 36, wherein said method comprises detecting the nucleic acid encoding PAWR using a PAWR probe.

38. The method of claim 37, wherein said PAWR probe comprises at least 10 nucleotides of the sequence 5′-AGTACGAAGATGATGAAGCAGGGC-3′ (SEQ ID NO: 9).

39. The method of claim 38, wherein said PAWR probe comprises at least 10 contiguous nucleotides of the sequence 5′-AGTACGAAGATGATGAAGCAGGGC-3′ (SEQ ID NO: 9).

40. The method of claim 39, wherein said PAWR probe comprises the sequence 5′-AGTACGAAGATGATGAAGCAGGGC-3′ (SEQ ID NO: 9).

41. The method of any one of claims 29 to 40, wherein said method further comprises normalizing the level of expression of PAWR based on the level of expression of a housekeeping gene.

42. The method of claim 41, wherein said housekeeping gene is ABL1.

43. The method of claim 42, wherein said normalization is performed according to the method defined in any one of claims 17 to 27.

44. The method of any one of claims 1 to 43, wherein said biological sample comprises nucleic acids obtained from peripheral blood cells or bone marrow cells from said patient.

45. The method of any one of claims 1 to 44, wherein said poor disease prognosis comprises low probability of survival, low probability of achieving a complete remission after induction chemotherapy and/or high risk of relapse.

46. The method of any one of claims 1 to 45, wherein said AML is an intermediate-risk AML.

47. The method of claim 46, wherein said intermediate-risk is FLT3-ITD negative AML.

48. The method of any one of claims 1 to 47, wherein said patient is less than 60 years old.

49. A method for determining the likelihood that a subject suffers from TP53-mutant acute myeloid leukemia (TP53mut AML), said method comprising:
(i) determining the level of expression of at least one of the genes listed in Table 2A and/or Table 2B in a biological sample comprising leukemic cells from said subject:
(ii) comparing said level of expression to a reference level of expression; and
(iii) determining the likelihood that said subject suffers from TP53mut AML based on said comparison;
wherein a differential expression of said at least one gene in said biological sample relative to said reference level of expression is indicative that said subject has a high likelihood of suffering from TP53mut AML.

50. The method of claim 49, wherein said method comprises determining the level of expression of at least one of the genes listed in Table 2A in said biological sample, and wherein a higher expression of said at least one gene in said sample relative to said reference level of expression is indicative that said subject has a high likelihood of suffering from TP53mut AML.

51. The method of claim 49 or 50, wherein said the level of expression is measured at the nucleic acid level.

52. The method of claim 51, wherein said the level of expression is measured by RNA sequencing (RNAseq) or reverse transcription polymerase chain reaction (RT-PCR).

53. A method for treating an AML patient having a good or poor disease prognosis identified based on expression of HMGA2 in a biological sample comprising leukemic cells from said patient, said method comprising treating said patient with a suitable treatment regimen for good or poor prognosis AML.

54. The method of claim 53, wherein said AML patient having a good or poor disease prognosis is further identified based on expression of PAWR in said biological sample.

55. The method of claim 53 or 54, wherein said method further comprises performing the method defined in any one of claims 1 to 48 to identify said AML patient having a good or poor disease prognosis.

56. The method of any one of claims 53 to 55, wherein said patient has poor disease prognosis, and wherein said treatment regimen comprises stem cell or bone marrow transplantation.

57. An assay mixture for the prognosis of AML, the assay mixture comprising: (i) a biological sample comprising leukemic cells from a patient suffering from AML; and (ii) one or more reagents for measuring the level of expression of HMGA2 in the sample.

58. The assay mixture of claim 57, further comprising (iii) one or more reagents for measuring the level of expression of PAWR in the sample.

59. The assay mixture of claim 57 or 58, wherein said one or more reagents for measuring the level of expression of HMGA2 in the sample comprises one or more of the primers and/or probes defined in any one of claims 4 to 13.

60. The assay mixture of claim 49 or 50, wherein said one or more reagents for measuring the level of expression of PAWR in the sample comprises one or more of the primers and/or probes defined in any one of claims 31 to 40.

61. The assay mixture of any one of claims 57 to 60, further comprising (iv) one or more reagents for measuring the level of expression of a housekeeping gene in the sample.

62. The assay mixture of claim 61, wherein said housekeeping gene is ABL1.

63. The assay mixture of claim 62, wherein said one or more reagents for measuring the level of expression of ABL1 in the sample comprises one or more of the primers and/or probes defined in any one of claims 18 to 27.


SEQUENCE LISTING (13 sequences)

SEQ ID NO: 1 - DNA, 19 bp, Artificial Sequence
  Synthetic oligonucleotide
cacttcagcccagggacaa

SEQ ID NO: 2 - DNA, 20 bp, Artificial Sequence
  Synthetic oligonucleotide
ctcaccggttggttcttgct

SEQ ID NO: 3 - DNA, 21 bp, Artificial Sequence
  Synthetic oligonucleotide
ctcagaagagaggacgcggcc

SEQ ID NO: 4 - DNA, 31 bp, Artificial Sequence
  Synthetic oligonucleotide
tggagataacactctaagcataactaaaggt

SEQ ID NO: 5 - DNA, 21 bp, Artificial Sequence
  Synthetic oligonucleotide
gatgtagttgcttgggaccca

SEQ ID NO: 6 - DNA, 28 bp, Artificial Sequence
  Synthetic oligonucleotide
ccatttttggtttgggcttcacaccatt

SEQ ID NO: 7 - DNA, 18 bp, Artificial Sequence
  Synthetic oligonucleotide
tggtcaacatccctgccg

SEQ ID NO: 8 - DNA, 20 bp, Artificial Sequence
  Synthetic oligonucleotide
ttgcatcttctcgtttccgc

SEQ ID NO: 9 - DNA, 24 bp, Artificial Sequence
  Synthetic oligonucleotide
agtacgaagatgatgaagcagggc

SEQ ID NO: 10 - DNA, 2520 bp, Homo sapiens
  CDS (812)..(1141)
cttgaatcttggggcaggaactcagaaaacttccagcccgggcagcgcgcgcttggtgcaagactcaggagctagcagcccgtccccctccgactctccggtgccgccgctgcctgctcccgccaccctaggaggcgcggtgccacccactactctgtcctctgcctgtgctccgtgcccgaccctatcccggcggagtctccccatcctcctttgctttccgactgcccaaggcactttcaatctcaatctcttctctctctctctctctctctctctctctctctctctctctctctctctctctctcgcagggtggggggaagaggaggaggaattctttccccgcctaacatttcaagggacacaattcactccaagtctcttccctttccaagccgcttccgaagtgctcccggtgcccgcaactcctgatcccaacccgcgagaggagcctctgcgacctcaaagcctctcttccttctccctcgcttccctcctcctcttgctacctccacctccaccgccacctccacctccggcacccacccaccgccgccgccgccaccggcagcgcctcctcctctcctcctcctcctcccctcttctctttttggcagccgctggacgtccggtgttgatggtggcagcggcggcagcctaagcaacagcagccctcgcagcccgccagctcgcgctcgccccgccggcgtccccagccctatcacctcatctcccgaaaggtgctgggcagctccggggcggtcgaggcgaagcggctgcagcggcggtagcggcggcgggaggcaggatgagcgcacgcggtgagggcgcggggcagccgtccacttcagcccagggacaacctgccgccccagcgcctcagaagagaggacgcggccgccccaggaagcagcagcaagaaccaaccggtgagccctctcctaagagacccaggggaagacccaaaggcagcaaaaacaagagtccctctaaagcagctcaaaagaaagcagaagccactggagaaaaacggccaagaggcagacctaggaaatggccacaacaagttgttcagaagaagcctgctcaggaggaaactgaagagacatcctcacaagagtctgccgaagaggactagggggcgccaacgttcgatttctacctcagcagcagttggatcttttgaagggagaagacactgcagtgaccacttattctgtattgccatggtctttccactttcatctggggtggggtggggtggggtgggggaggggggggtggggtggggagaaatcacataaccttaaaaaggactatattaatcaccttctttgtaatcccttcacagtcccaggtttagtgaaaaactgctgtaaacacaggggacacagcttaacaatgcaacttttaattactgttttcttttttcttaacctactaatagtttgttgatctgataagcaagagtgggcgggtgagaaaaaccgaattgggtttagtcaatcactgcactgcatgcaaacaagaaacgtgtcacacttgtgacgtcgggcattcatataggaagaacgcggtgtgtaacactgtgtacacctcaaataccaccccaacccactccctgtagtgaatcctctgtttagaacaccaaagataaggactagatactactttctctttttcgtataatcttgtagacacttacttgatgatttttaactttttatttctaaatgagacgaaatgctgatgtatcctttcattcagctaacaaactagaaaaggttatgttcatttttcaaaaagggaagtaagcaaacaaatattgccaactcttctatttatggatatcacacatatcagcaggagtaataaatttactcacagcacttgttttcaggacaacacttcattttcaggaaatctacttcctacagagccaaaatgccatttagcaataaataacacttgtcagcctcagagcatttaaggaaactagacaagtaaaattatcctctttgtaatttaatgaaaaggtacaacagaataatgcatgatgaactcacctaattatgaggtgggaggagcgaaatctaaatttcttttgctatagttatacatcaatttaaaaagcaaaaaaaaaaaaggggggggcaatctctctctgtgtctttctctctctctcttcctctccctctctcttttcattgtgtatcagtttccatgaaagacctgaataccacttacctcaaattaagcatatgtgttacttcaagtaatacgttttgacataagatggttgaccaaggtgcttttcttcggcttgagttcaccatctcttcattcaaactgcacttttagccagagatgcaatatatccccactactcaatactacctctgaatgttacaacgaatttacagtctagtacttattacatgctgctatacacaagcaatgcaagaaaaaaacttactgggtaggtgattctaatcatctgcagttctttttgtacac

SEQ ID NO: 11 - protein, 109 aa, Homo sapiens
MSARGEGAGQPSTSAQGQPAAPAPQKRGRGRPRKQQQEPTGEPSPKRPRGRPKGSKNKSPSKAAQKKAEATGEKRPRGRPRKWPQQVVQKKPAQEETEETSSQESAEED

SEQ ID NO: 12 - DNA, 1967 bp, Homo sapiens
  CDS (287)..(1309)
ctgtcctgggattgcctggagctccgcaccgcgagtttgccgcggcactttccgcgcggcggaagagcgcgcgccagcttcggcacacctgggagccggatcccagccctacgcctcgtcccctacaagctcctccaagccccgccggctgctgtgggagcggcggccgtccctctcctggaggtcgtctcctggcatcctcggggccgcaggaaggaagaggaggcagcggccggagccctggtgggcggcctgaggtgagagcccgaccggcccctttgggaatatggcgaccggtggctaccggaccagcagcggcctcggcggcagcaccacagacttcctggaggagtggaaggcgaaacgcgagaagatgcgcgccaagcagaaccccccgggcccggcccccccgggagggggcagcagcgacgccgctgggaagccccccgcgggggctctgggcaccccggcggccgccgctgccaacgagctcaacaacaacctcccgggcggcgcgccggccgcacctgccgtccccggtcccgggggcgtgaactgcgcggtcggctccgccatgctgacgcgggcggcccccggcccgcggcggtcggaggacgagcccccagccgcctctgcctcggctgcaccgccgccccagcgtgacgaggaggagccggacggcgtcccagagaagggcaagagctcgggccccagtgccaggaaaggcaaggggcagatcgagaagaggaagctgcgggagaagcggcgctccaccggcgtggtcaacatccctgccgcagagtgcttagatgagtacgaagatgatgaagcagggcagaaagagcggaaacgagaagatgcaattacacaacagaacactattcagaatgaagctgtaaacttactagatccaggcagttcctatctgctacaggagccacctagaacagtttcaggcagatataaaagcacaaccagtgtctctgaagaagatgtctcaagtagatattctcgaacagatagaagtgggttccctagatataacagggatgcaaatgtttcaggtactctggtttcaagtagcacactggaaaagaaaattgaagatcttgaaaaggaagtagtaagagaaagacaagaaaacctaagacttgtgagactgatgcaagataaagaggaaatgattggaaaactcaaagaagaaattgatttattaaatagagacctagatgacatagaagatgaaaatgaacagctaaagcaggaaaataaaactcttttgaaagttgtgggtcagctgaccaggtagaggattcaagactcaatgtggaaaaaatattttaaactactgattgaatgttaatggtcaatgctagcacaatattcctatgctgcaatacattaaaataactaagcaagtatatttatttctagcaaacagatgtttgttttcaaaatacttctttttcattattggttttaaaaaagcattatccttttatctcacaaataagtaatatctttcagttattaaatgatagataatgcctttttggttttgtgtggtattcaactaatacatggtttaaagtcacagccgtttgaatatattttatcttggtagtacattttctcccttaggaatatacatagtctttgtttacatgagttcaaatacttttgggatgttaccttcacatgtcctattactgatgtgtgcaaccttttatgtgttgatgactcactcataaaggtttttgtctactgtcatttgttctttccacttattctaagcatttagagtaatagagtcatacttttttataacagcaaccttttaaaaggaaagctcttataaagtcactgtcatgttttagttgactaaatataaatttaagagaatacttgaattgtgctatagtaaataaaaatttactattttgtgtttgaaaaaaaaaaaaaaaaaa

SEQ ID NO: 13 - protein, 340 aa, Homo sapiens
MATGGYRTSSGLGGSTTDFLEEWKAKREKMRAKQNPPGPAPPGGGSSDAAGKPPAGALGTPAAAAANELNNNLPGGAPAAPAVPGPGGVNCAVGSAMLTRAAPGPRRSEDEPPAASASAAPPPQRDEEEPDGVPEKGKSSGPSARKGKGQIEKRKLREKRRSTGVVNIPAAECLDEYEDDEAGQKERKREDAITQQNTIQNEAVNLLDPGSSYLLQEPPRTVSGRYKSTTSVSEEDVSSRYSRTDRSGFPRYNRDANVSGTLVSSSTLEKKIEDLEKEVVRERQENLRLVRLMQDKEEMIGKLKEEIDLLNRDLDDIEDENEQLKQENKTLLKVVGQLTR